(19) United States
(12) Patent Application Publication
(10) Pub. No.: US 2021/0246518 A1
(43) Pub. Date: Aug. 12, 2021

(54) Method for Detecting in a Cell RNA-Protein Interaction

(71) Applicants: Centre National de la Recherche Scientifique, Paris (FR); Institut Naitonal de la Sante et de la Recherche Medicale, Paris (FR); Institut Curie, Paris (FR)

(72) Inventor: Alena Shkumatava, Paris (FR)

(73) Assignees: Centre National de la Recherche Scientifique, Paris (FR); Institut Naitonal de la Sante et de la Recherche Medicale, Paris (FR); Institut Curie, Paris (FR)

(21) Appl. No.: 17/273,402

(22) PCT Filed: Sep. 9, 2019
(86) PCT No.: PCT/EP2019/073999

§ 371 Date: Mar. 4, 2021

(30) Foreign Application Priority Data
Sep. 10, 2018 (EP) 18306185.2

(51) Int. Cl. C12Q 1/6897; C12N 15/10

(52) U.S. Cl. CPC C12Q 1/6897; C12N 15/1055

(57) ABSTRACT

The present invention relates to a method for detecting the interaction in a cell between a RNA and a protein, wherein the cell expresses: —the RNA fused to a hairpin, called hairpin-tagged RNA, —the protein fused to a tag, called tagged-protein, —a reporter protein fused to a protein which binds to said hairpin, wherein said reporter protein is a luminescent reporter protein, said method comprises the lysis of the cell and the detection of the complex tagged protein/hairpin-tagged RNA/hairpin binding protein—reporter protein.

### FIELD OF THE INVENTION

The present invention relates to a method for detecting the interaction between an RNA and a protein comprising the expression in a cell of the RNA fused to a hairpin, the protein fused to a tag, and a reporter protein fused to a hairpin binding protein.

### BACKGROUND OF THE INVENTION

Regulatory RNAs are essential determinants of development, physiology and disease. Because RNAs exert their cellular function through RNA-binding proteins (RBPs), the identification of RNA-protein interactions is key for a molecular understanding of this class of regulatory molecules. The identification of RNA-protein interactions is essential to decipher the cellular functions and molecular mechanisms of action of regulatory RNAs that typically act through the formation of dynamic ribonucleoprotein (RNP) complexes (Engreitz et al., 2016).

To date, RNA purification followed by mass spectrometry is used to identify RNA-bound proteins.

There are several established protein-centered approaches such as cross-linking immunoprecipitation (CLIP) methods that reliably identify RNAs bound by individual proteins (Konig et al., 2012). By contrast, when aiming to determine proteomes bound by individual transcripts, RNA-centered approaches are applied that are mainly based on affinity purification of an RNA of interest followed by mass-spectrometry identification of the co-purified proteins (AP-MS) (Chu et al., 2015a).

While these methods have been widely applied and have led to the identification of functionally relevant RNA interactors, they have limitations. First, AP-MS approaches require scaling up the cellular material due to the general low efficiency of RNA affinity purifications. As such, when applied to RNAs expressed at the low copy number characteristic of many long noncoding RNAs (lncRNAs) (Derrien et al., 2012), generating sufficient cellular material for AP-MS is challenging and may increase the risk of contamination by non-specific RBPs. Second, RBPs that are expressed at relatively low levels often do not reach a threshold required for the robust detection by mass spectrometry. Third, AP-MS approaches capture stable RNP complexes, possibly unable to detect functionally important transient RNA-protein interactions. Finally, most currently available approaches enable identification of proteins binding the full-length transcript of interest, whereas many lncRNAs have a modular organization with discrete RNA regions performing different functions (Engreitz et al., 2016; Guttman and Rinn, 2012).

Several in vivo methods have been used to analyze RNA-protein interactions including FRET-based and in vivo imaging-based methods (Huranova et al, 2009. RNA, 15(11), 2063-2071; Han et al., 2014 NAR, 42(13),e103). Such methods are however not suitable for the comprehensive identification of binding proteins of a given RNA of interest because they can only measure molecules that are located in the close proximity (<10 nm; while mRNA and lncRNA molecules are very large and typically exceed by far this small distance in their size). Importantly, FRET only measures proximity of two target biomolecules but does not report if they actually interact with each other. As such, the methods above are not suitable for the unbiased identification of true RNA-protein binding and detection of interactions that occur at a distance >10 nm. Thus, there is still a need to develop a method being easy to implement in living cells and which enables detection of RNA-protein interactions, in particular when the interaction is transient and/or when the RNA is low expressed, such as the interaction between lncRNAs and proteins.

### SUMMARY

A first aspect of the invention relates to a method for detecting the interaction in a cell between an RNA and a protein, wherein the cell expresses:
the RNA fused to a hairpin, called hairpin-tagged RNA,
the protein fused to a tag, called tagged-protein,
a protein which binds to said hairpin, called hairpin binding protein, fused to a reporter protein,

wherein said reporter protein is a luminescent reporter protein, said method comprises the lysis of the cell and the detection of the complex tagged protein/hairpin-tagged RNA/hairpin binding protein—reporter protein.

A second aspect of the invention relates to the use of the method for detecting the effect of a compound on the interaction in a cell between an RNA and a protein.

A third aspect of the invention relates to the use of the method for detecting the interaction in a cell between two RNAs and a protein.

A fourth aspect of the invention relates to the use of the method for determining the effect of the RNA methylation on the interaction between an RNA and a protein.

Definition

According to the present application, the RNA is fused to a hairpin. The whole is called hairpin-tagged RNA. The terms “hairpin tag” and “hairpin” are used indifferently.

In particular, the hairpin used to tag the RNA is selected from the group consisting in: MS2, PP7, AN, TAR, iron responsive elements (IREs) and U1A hpII.

In particular, the RNA is fused to a plurality of identical consecutive hairpins, preferably with 2 to 24 consecutive identical hairpins, more preferably with 2 to 10, more preferably with 4 to 8, more preferably with 10 consecutive identical hairpins.

According to the present application, the protein is fused to a tag. The whole is called tagged-protein. Typically the tag should be selected to avoid steric hindrance that might affect proper functioning and folding of the protein in vivo. Thus the selected tag is preferably of small size in particular less than 10 000 Da, notably less than 5000 Da in particular about 3500 Da or less. Typically also the tag is an antigenic molecule, notably an artificial antigen that can be recognized by an antigen binding member (typically an antibody or a variant thereof as classically used in the field). For example the tag should be recognizable in an ELISA-like assay.

In particular, the tag used to obtain the tagged-protein is selected from the group consisting in: FLAG, HIS, CBP, HA, Myc, poly His, V5 and combination thereof. In some embodiments, one or more identical or different tags can be used as per the present disclosure. In particular, the protein is fused to a plurality of identical consecutive tags, preferably with 2 to 5 identical consecutive tags, more preferably with 3 identical consecutive tags.

According to the present application, a protein which binds to the hairpin of the hairpin-tagged RNA, is fused to a luminescent reporter protein. This protein is also called hairpin binding protein.

In particular, the dissociation constant of the complex hairpin/hairpin binding protein is between 10⁻⁷ and 10⁻¹³ M.

In particular, the hairpin binding protein is selected from the group consisting in: MS2CP (MS2 coat protein), PP7CP (PP7 coat protein), Qβ, GA, BoxB, TAT, IRP and U1A.

In particular, the hairpin used to tag the RNA and the hairpin binding protein are selected from the couples hairpin/binding protein consisting in: MS2/MS2CP, PP7/PP7CP, MS2/Qβ, MS2/GA, AN/BoxB, TAR/TAT, iron responsive elements (IREs)/IRP, and U1A hpII/U1A.

According to the present application, the reporter protein is a luminescent protein.

In particular, the luminescent reporter protein is an enzyme which catalyzed a bioluminescence reaction. Typically, the luminescent reporter protein is a luciferase. In particular, the luciferase is selected among the group consisting in: NanoLuciferase, FLuciferase North American firefly luciferase, Japanese firefly (Genji-botaru) luciferase, Italian firefly luciferase, Japanese firefly (Heike) luciferase, East european firefly luciferase, Pennsylvania firefly luciferase, Railroad worm luciferase, Renilla luciferase (Rluc), Green Renilla luciferase, Gaussia luciferase, Gaussia-Dura luciferase, Cypridina luciferase, Metridia luciferase and OLuc. In particular, the luciferase is the NanoLuciferase or the FLuciferase.

In particular, the luminescent reporter protein is a fluorescent protein. Typically, the reporter protein can be a green fluorescent protein (GFP), a yellow fluorescent protein (YFP), a cyan fluorescent protein (CFP) or a blue fluorescent protein (BFP).

According to the present application, the term “complex” designates the complex tagged protein/ hairpin-tagged RNA/hairpin binding protein—reporter protein.

IncPRINT METHOD

The present invention concerns a method which allows the detection of the interaction between any given RNA and any given protein.

The present invention relates to a method for detecting the interaction in a cell between an RNA and a protein, wherein the cell expresses:
the RNA fused to a hairpin, called hairpin-tagged RNA,
the protein fused to a tag, called tagged-protein,
a protein which binds to said hairpin, called hairpin binding protein, fused to a reporter protein,

wherein the reporter protein is a luminescent protein, and wherein said method comprises the lysis of the cell and the detection of the complex tagged protein/hairpin-tagged RNA/hairpin binding protein—reporter protein.

As demonstrated in the result included in the present application, this method is a sensitive and easy to implement method. In fact, the method enables identification of proteins associated with RNA expressed at low endogenous levels such as lncRNAs.

The method allows the detection of transient interactions, and dispenses detection with the use of UV detector, mass spectrometer, or chemical crosslinking.

The method is also a reliable high-throughput method to systematically and quantitatively detect RNA-protein interactions in living cells.

The present method is not only (1) a small-scale method for detection of protein-RNA interactions that typically allows to validate already known interactions but (2) also further allows easily identifying novel interactions by profiling thousands of proteins against one test RNA

Preferably, the cell is a mammalian cell, more preferably a cell of a human cell line.

This method, called incPRINT (in cell protein-RNA interaction), is based on the co-expression of a hairpin-tagged RNA and a tagged protein in a cell, said cell expressing a luminescent reporter protein fused to a protein which binds to said hairpin. The interaction between the RNA and the protein on one side, and between the hairpin tag and the hairpin binding protein on another side, leads to the formation of a complex tagged protein/hairpin-tagged RNA/hairpin binding protein—reporter protein (FIG. 1).

After cell lysis, this complex is detected in vitro to highlight the interaction between the RNA and the protein.

In particular, indirect RNA-protein interactions bridged by DNA are eliminated by DNAse treatment after the cell lysis step.

Previous to the detection and after cell lysis, the complex is typically isolated on a solid support using a ligand which binds specifically to the tag of the tagged protein. Typically, the solid support is constituted by beads or wells. In particular, the complex is isolated by immunoprecipitation using an antibody directed against the tag, as a ligand.

The detection of the complex is then carried out by detecting the luminescent reporter protein and detecting the tag of the tagged protein. The both detections indicate the interaction between the RNA and the protein.

Typically, when the reporter protein is an enzyme which catalyzed a bioluminescence reaction, such as luciferase, the detection of the luminescent reporter protein is made by bioluminescence measurement.

Typically, when the reporter protein is a fluorescent protein, the detection of the luminescent reporter protein is made by fluorescence measurement.

Typically, the detection of the tag of the tagged protein is carried out by an ELISA-like assay, using an antigen binding molecule, such as antibody, directed against the tag.

To detect the RNA-protein interaction in a quantitative manner, the amount of the complex is measured by the signal of the luminescent reporter protein. Indirect RNA-protein interactions bridged by DNA are eliminated by DNAse treatment after the cell lysis step. To control for protein expression levels, the abundance of the tagged protein is measured by ELISA using a second antibody directed against the protein tag.

Typically, to obtain a cell expressing:
the RNA fused to a hairpin, called hairpin-tagged RNA,
the protein fused to a tag, called tagged-protein,
a protein which binds to said hairpin, called hairpin binding protein, fused to a luminescent reporter protein,

a cell expressing the luminescent reporter protein fused to a hairpin binding protein, is transfected by two vectors which respectively express the hairpin-tagged RNA and the tagged protein;

In particular, the lysis of the cell is carried out between 24 and 72 h, after the transfection, in particular after 48 h.

The incPRINT method is flexible in scale and can be used either as a low- or high-throughput method.

The inventors have tested the incPRINT method on a library of about 3000 human tagged proteins including about 1500 all known RBPs (based on (Baltz et al., 2012; Castello et al., 2012)), about 1300 transcription factors (Taipale et al., 2012) and about 170 chromatin-associated proteins. By interrogating this protein library, they identified in a high-throughput manner cellular RNA-protein interactions.

The Inventors have proved that their RNA-centric method called incPRINT allowed to systematically detect and quantitatively measure RNA-protein interactions in cells using luminescence and can be used in a high-throughput way.

Thus, in one embodiment the method allows screening a library of vectors expressing proteins, to identify the proteins interacting with a target RNA.

The present invention also relates to a method for identifying the proteins interacting with a target RNA, wherein cells expressing the luminescent reporter protein fused to a hairpin binding protein, are transfected by a vector expressing the hairpin-tagged RNA and each of the cells is transfected by a vector expressing a tagged protein from a protein library.

As a proof-of-principle, the inventors applied the high-throughput method to Xist, a long non-coding RNA that is essential for X-chromosome inactivation (XCI) in mammals, more particularly to three conserved regions of Xist that carried out different functions during XCI. They identified and quantified systematically the interactions of these three regions of Xist with a custom library of about 3000 proteins including the majority of human RNA-binding proteins, epigenetic and transcription factors and chromatin modifiers. By the incPRINT method, they identified both previously known proteins associated with functionally distinct regions of Xist and new Xist-associated proteins required for XCI, that evaded detection with previous approaches. Moreover, they showed that the majority of the RNA-protein interactions defined by incPRINT are RNA region-specific. Furthermore, they demonstrated that two of the newly identified RBPs are required for the proper initiation of XCI.

The inventors also applied the method to the lncRNA Firre which has an endogenous expression level of approximately 20 molecules per cell. They identified RBPs specifically interacting with this IncRNA, thus demonstrating that the incPRINT is applicable to any given RNA including transcripts expressed at low endogenous levels.

Applied to long noncoding RNAs (lncRNAs) as a proof-of concept, incPRINT reliably identified both previously known and novel functional lncRNA-protein interactions that evaded detection with other approaches, highlighting incPRINT's potential for discovery. The method enables assignment of RNA binding proteins to defined regions of a long full-length transcript and detected highly RNA sequence-specific protein interactions. This feature of is particularly advantageous for defining the RNA region-specific binding proteome of large modular RNAs with multiple functional regions such as the ˜17 kb long Xist transcript.

Using a series of control experiments, the inventors demonstrated the robustness of incPRINT, its throughput scalability and reproducibility. Importantly, they showed that the reporter protein signal detected by incPRINT is RNA-dependent and the interactions are formed in living cells prior to cell lysis.

The incPRINT method overcomes several limitations that are associated with other techniques and is particularly suitable for quantifying the interactions between an RNA of interest with thousands of proteins via a simple scalable assay.

Thus, in another embodiment the method allows screening a library of vectors expressing RNAs, to identify the RNAs interacting with a target protein.

The present invention also relates to a method for identifying the RNAs interacting with a target protein, wherein cells expressing the luminescent reporter protein fused to a hairpin binding protein, are transfected by a vector expressing the tagged protein and each of the cells is transfected by a vector expressing a hairpin-tagged RNA from an RNA library.

Drug Discovery

In one embodiment, the method is used for identifying the effect of a compound on the RNA -protein interactions (FIG. 7).

The present invention also relates to a method for detecting in a cell the effect of a compound on the interaction between an RNA and a protein, said method comprising the implementation of the incPRINT method, previously described, separately into a cell cultivated in the presence of a compound to be tested and into a cell cultivated in the absence of said compound in the culture medium.

Thus, the present invention also concerns a method for detecting in a cell the effect of a compound on the interaction between an RNA of interest and a protein of interest, said method incPRINT being carried out as previously described in absence and in presence of a compound in the culture medium of the cell.

In particular, the lysis of the cell is carried out between 24 h and 72 h after the addition of the compound, in particular 48 h.

In the particular embodiment wherein a cell expressing the luminescent reporter protein fused to a hairpin binding protein, is transfected by two vectors which respectively express the hairpin-tagged RNA and the tagged protein, the compound is added in the culture medium between 12 h and 36 h after the transfection, preferably 24 h after the transfection.

Thus, in this embodiment, the detection of the complex tagged protein/hairpin-tagged RNA/ hairpin binding protein—reporter protein is carried out in the absence and in the presence of a compound to be tested, and then the obtained results are compared to identifying the effect of a compound on the RNA-protein interactions.

Changes of the complex detection in presence of the compound indicate an effect of said compound on the interaction between the RNA of interest and the protein of interest. A diminution of the detection compared to the one measured in the absence of the compound, indicates an inhibition of the interaction RNA-protein by the compound. At the opposite, an increase of the detection compared to the one measured in the absence of the compound, indicates an increase of the interaction between the RNA of interest and the protein of interest by the presence of the compound.

It is well-understood that no change indicates that the compound has no effect on the interaction between the RNA of interest and the protein of interest.

To control the compound toxicity, a cell viability test (for example by RealTime-Glo MT Cell Viability Assay, Promega) is performed upon compound application, prior to the cell lysis. To control the specificity of each tested compound, protein and RNA levels are tested by ELISA and qPCR, respectively.

It is possible to apply different concentrations of the compound enabling simultaneous profiling of the drug efficiency on the RNA-protein interaction and cell viability.

This embodiment of the method is flexible in scale and can be used either as a low- or high-throughput method.

In addition to the advantages of the method incPRINT, this particular embodiment enables a high-throughput identification of drugs such as small molecule inhibitors targeting RNA-protein interactions in cells. Moreover, it allows to simultaneous testing the effect of the compound on the interaction RNA-protein and the cell viability upon compound application.

The present invention also relates to a method for identifying the proteins interacting with a target RNA, wherein cells expressing the luminescent reporter protein fused to a hairpin binding protein, are transfected by a vector expressing the hairpin-tagged RNA and each of the cells is transfected by a vector expressing a tagged protein from a protein library.

The present invention also relates to a method for screening in cells the effect of compounds on the interaction between an RNA of interest and a protein of interest, said method incPRINT being carried out as previously described, in absence of a compound in the culture medium and separately in the presence of a different compound.

Using in a high-throughput way by carrying out individual assay with each compound of a chemical bank, the present method enables a drug screening targeting RNA-protein interactions, such as small inhibitors targeting RNA-protein interactions in living cells.

For example, the method enables the discovery of drugs to target and inhibit the function of pathogenic RNA molecules such as viral RNA, toxic neurogenerative RNA, stress RNA.

It allows simultaneously identifying new compounds, testing the cell viability and testing other cellular parameters such as cell metabolism.

Dual Interaction RNA-Protein (Dual incPRINT)

In one embodiment, the method is used for detecting the interaction in a cell between two RNAs and a protein (FIG. 8).

The present invention also relates to a method for detecting the interaction in a cell between two RNAs and a protein, said method comprises two hairpin-tagged RNAs, each of the RNAs being fused to a different hairpin, and wherein said cell expresses two different reporter proteins respectively fused to two different proteins which respectively bind to said hairpins, said method being implemented as the incPRINT method previously described.

Thus, in this embodiment, the method incPRINT described above is carried out in a cell expressing two hairpin-tagged RNAs and a tagged protein, said cell also expressing two different luminescent reporter proteins respectively fused to two different proteins which binds to said hairpins.

The detection of the tag of the tagged protein and the detection of the luminescent reporter protein allows to identify which of the two RNA interacts with the protein.

Each of the two luminescent reporter proteins being fused to a different hairpin binding protein, and each of these proteins binding respectively to each hairpin tag fused to each of the two RNAs, the detection of the tag of the tagged protein and the detection of the luminescent reporter protein allows to identify with which RNA, the protein interacts.

In the case of the detection of both luminescent reporter proteins, the expression levels of the two RNAs have to be measured by qPCR, to control that both RNAs are expressed at equal levels. If it is the case, the detection of both luminescent reporter proteins indicates that each of the two RNAs is able to interact with the protein.

The dual-incPRINT method allows to simultaneous profile the RNA-protein interactions for two RNAs, thanks to two different luminescent reporter proteins.

In a particular embodiment, the two RNAs are two versions of the same transcript, one is the RNA normally expressed by cells, and the other a mutated version of said RNA. Thus, this method allows to identify in a same cell, the effect of an RNA mutation on the interaction RNA-protein.

In addition to the advantages of the method incPRINT, this embodiment of the method based on expressing in the same cell two different RNAs and two different luminescent reporter proteins, allows simultaneous to profile the RNA-protein interactions for two RNAs.

This method enables the characterization of the interaction between a protein and two different RNAs in the same cell, leading to a cost- and time-benefit. It allows the between a mutated and «normal» version of the same RNA in the same cell. It is also possible to increases robustness of the method if one does it with the same RNA tagged by two different tags.

In particular, the present invention concerns a method for detecting the interaction in a cell between two RNAs and a protein, wherein the cell expresses:
two RNAs respectively fused to two different hairpins, called hairpin-tagged RNAs,
the protein fused to a tag, called tagged-protein,
two reporter proteins respectively fused to two different proteins which binds to said hairpins,

wherein said reporter proteins are luminescent reporter proteins, said method comprises the lysis of the cell and the detection of the complexes tagged protein/hairpin-tagged RNA/hairpin binding protein—reporter protein.

In a particular embodiment, the hairpin tags used to label the two RNAs are MS2 and PP7, and the corresponding hairpin binding proteins are MS2 coat protein (MS2CP) and PP7 coat protein (PP7CP). These proteins are expressed in the cell in fusion with respectively the two different luminescent reporter proteins.

In particular, the two luminescent reporter proteins are luciferases, more particularly Nanoluciferase (Nluc) and Firefly luciferase, also called Fluciferase (Fluc). More particularly, the cell expresses MS2CP fused to Nluc and PP7CT fused to Fluc.

In a preferred embodiment, one of the two RNAs is fused with 10 consecutive MS2 hairpin tags.

In a particular embodiment, the protein is tagged with a FLAG tag. Thus, the antibody used to the immunoprecipitation of the complex tag-protein-RNA-reporter protein is an antibody anti-FLAG.

In a particular embodiment, the protein is fused with a plurality of consecutive FLAG tags. Preferably, it is fused with 3 consecutive FLAG tags.

Thus, in the particular embodiment where the cell expresses MS2CP fused to Nluc and PP7CT fused to Fluc, the detection of the FLAG tag and the detection of the Nluc indicate that the RNA which binds to the protein is the one fused to hairpin tag MS2. Similarly, and the detection of the FLAG tag and the detection of the Fluc indicate that the RNA which binds to the protein is the one fused to hairpin tag PP7.

In a particular embodiment, said method comprises:
a cell expressing the two luminescent reporter proteins, each fused to a protein which binds specifically to the hairpin of one of the hairpin-tagged RNA transfected by three vectors which respectively express a first hairpin-tagged RNA, a second hairpin-tagged RNA and a tagged protein;
the lysis of the cell;
the detection of the complexes tagged protein/hairpin-tagged RNA/hairpin binding protein—reporter protein.

In another embodiment, the method is used for detecting the interaction in a cell between two proteins and an RNA.

In this embodiment, the method incPRINT described above is carried out in a cell expressing two tagged proteins, each one being tagged by a different tag, and a hairpin-tagged RNA, said cell expressing a luminescent reporter protein fused to a protein which binds to said hairpin.

The detection of the tag of the tagged protein and the detection of the luminescent reporter protein allows to identify which of the two proteins interacts with the RNA.

In the case of the detection of both tags of the tagged proteins, the expression levels of the two proteins have to be measured by ELISA, to control that both proteins are expressed at equal levels. If it is the case, the detection of both tags of the tagged proteins indicates that each of the two proteins is able to interact with the RNA.

The dual-incPRINT method allows to simultaneous profile the RNA-protein interactions for two proteins.

In a particular embodiment, the two proteins are two versions of the same protein, one is the protein normally expressed by cells, and the other a mutated version of the protein. Thus, this method allows to identify in a same cell, the effect of a protein mutation on the interaction RNA-protein.

In addition to the advantages of the method incPRINT, this embodiment of the method based on expressing in the same cell two different proteins respectively tagged with a different tag, allows simultaneous to profile the RNA-protein interactions for two proteins.

Thus, this method enables the characterization of the interaction between two proteins and an RNA in the same cell, which decreases the cost and time.

RNA Methylation (Epi-incPRINT)

In another embodiment, the method is used for determining the effect of the RNA methylation on the interaction between an RNA and a protein (FIG. 9).

Thus, the present invention also relates to a method for determining the effect of the RNA methylation on the interaction between an RNA and a protein, wherein said method being carried out according to the incPRINT method described above, respectively into a cell having a normal profile for the RNA methylation and into a cell comprising the inactivation of one or more genes involved in the RNA methylation.

In particular, the cells are cells from a same cell line, one having a normal profile for the RNA methylation and the other comprising the inactivation of one or more genes involved in the RNA methylation.

A cell having a normal profile for the RNA methylation means that this cell has no genetic modifications of genes involved in the RNA methylation.

A gene which is inactivated means that the gene is no longer expressed. Typically, a gene is inactivated by deletion. In particular, the gene deletion is obtained by genetic knock-out, more particularly using CRISPR-Cas9.

The comparison of the detection of the complex between the two cells allows to identify the effect RNA methylation on the RNA-protein interaction.

The absence of detection of the complex in the cell comprising inactivation for one or more genes involved in the RNA methylation, indicates that methylation of the RNA has an effect on the interaction RNA-protein.

By contrast, the detection of the complex in the cell comprising inactivation for one or more genes involved in the RNA methylation, indicates that methylation of the RNA has no effect on the interaction RNA-protein.

In particular, the cell comprising the inactivation of one or more genes involved in the RNA methylation has inactivation for one or more genes involved in the methylation of the adenosine base at the nitrogen-6 position (m⁶A) in the RNA. More particularly, the cell is a mammalian cell wherein the genes Mettl3, Mettl14 or Wtap, more particularly for the genes Mettl3 and Mettl14, are inactivated. Genes responsible of the methylation m⁶A are well-known by the art (Batista et al., 2014, Cell Stem Cell (PMID: 25456834); Schwartz et al., 2014, Cell Reports (PMID: 24981863); Liu, J., et al. (2014). A METTL3-METTL14 complex mediates mammalian nuclear RNA N6-adenosine methylation. Nat. Chem. Biol. 10, 93-95; Ping, X. L., et al. (2014). Mammalian WTAP is a regulatory subunit of the RNA N6-methyladenosine methyltransferase. Cell Res. 24, 177-189).

The cell deleted for the expression of the genes involved in m⁶A RNA modification is called m⁶A-depleted cell and the cell having a normal profile for the RNA methylation is called m⁶A positive cell.

In one embodiment, the present invention relates to a method for determining the effect of the RNA methylation on the interaction between an RNA and a protein, said method being carried out according to the incPRINT method described above, and said method being carried out respectively into two mammalian cells from a same cell line, one comprising the inactivation of the genes involved in the RNA methylation and the other having no genetic modification for genes involved in the RNA methylation.

In particular, the cell comprising the inactivation of the genes involved in the RNA methylation, comprises the deletion of the genes Mettl3 and Mettl14.

### FIGURES

FIG. 1: Principle of the incPRINT method.

HEK293 cells stably expressing a NanoLuciferase-MS2CP recombinant protein were co-transfected with a test MS2-tagged RNA and a test FLAG-tagged protein.

RNA-protein complexes were formed in transfected cells.

Transfected cells were lysed to immuno-purify RNP complexes.

Individual RNP complexes were immobilized in plates coated with the anti-FLAG antibody. After washing off nonspecific proteins, the FLAG-protein-RNA-Luciferase complexes were detected by luminescence.

Expression levels of the FLAG-tagged test proteins were detected by ELISA using another anti-FLAG antibody.

FIG. 2: incPRINT measures cellular RNA-protein interactions.

(A) Interaction intensities detected between Xist(A)-MS2 and the indicated factors, with and without RNAase treatment. Data from two biological replicates are presented as mean±SD. RLU are Relative Light Units.

(B) Interaction intensities between Xist(A)-MS2 and the indicated factors. In cell interaction values were measured in a standard incPRINT experiment. In vitro interaction values resulted from separate transfections of the tagged-proteins and Xist(A)-MS2 RNA, which were combined for interaction analyses after the cell lyses. Data from two biological replicates for each experiment are presented as mean±SD. RLU are Relative Light Units.

(C) Scatter plot showing correlation of the Xist(A)-MS2 interaction intensities determined by NanoLuc luciferase luminescence from two biological replicates. Shown are median-normalized values. Squared Pearson correlation coefficient is indicated.

(D) Scatter plot showing correlation of FLAG-tagged protein expression levels determined by anti-FLAG ELISA from two biological replicates. Shown are median-normalized values. Squared Pearson correlation coefficient is indicated.

(E) Scatter plot showing RNA-protein interaction intensities and protein expression levels. RLU are Relative Light Units.

FIG. 3: incPRINT identifies Firre—interacting proteins

(A) Scatter plot showing Firre-MS2 interaction intensities as an average from two biological replicates plotted against the interaction score enrichment of Firre—MS2 over MS2 alone. The vertical dotted line delineates a threshold set at 2 normalized RLU to define Firre-MS2 interacting proteins. White dots are proteins that do not bind to neither Firre-MS2 nor to MS2 alone; black dots are Firre-interacting proteins. Selected proteins known to interact with Firre are indicated. See also experimental procedures for data normalization. RLU are Relative Light Units.

(B) RNA binding domain analyses.

(C)-(D) incPRINT with different concentrations of Firre-MS2. (C) Interaction intensities detected between the indicated proteins and Firre-MS2 expressed at different levels, whereas the 1:50 dilution corresponds to the endogenous FIRRE expression levels in HEK293T cells. Data from two biological replicates are presented as mean±SD. RLU are Relative Light Units. (D) Expression levels of the indicated FLAG-tagged proteins measured by ELISA. Standard incPRINT protein concentration was used for all tested conditions. Data from two biological replicates are presented as mean±SD. RLU are Relative Light Units.

FIG. 4: Differential sets of proteins interact with A-, F- and C-repeat regions of Xist

(A) Schematic representation of the mouse Xist transcript and its A- to F-conserved repeat regions. Exons are indicated as boxes, introns as lines. A zoom-in image shows the 5′ region of Xist. The horizontal color bars indicate the position of Xist fragments along the Xist transcript used in the incPRINT experiments. 0.9 kb, 2 kb, and 1,7 kb fragments for Xist(A), Xist(F) and Xist(C) were used, respectively.

(B) Scatter plot showing Xist(A)-MS2 interaction intensities (average from two biological replicates) plotted against the interaction score enrichment of Xist(A)-MS2 over MS2 alone. The vertical dotted line delineates a threshold set at 2 normalized RLU to define Xist(A)-MS2 interacting proteins. White dots are proteins that do not bind to neither Xist(A)-MS2 nor to MS2 alone; black dots are proteins defined as Xist(A)-MS2 binders. See also experimental procedures for data normalization. RLU are Relative Light Units.

(C) Scatter plot showing Xist(F)-MS2 interaction intensities (average from two biological replicates) plotted against the interaction score enrichment of Xist(F)-MS2 over MS2 alone. The vertical dotted line delineates a threshold set at 2 normalized RLU to define Xist(F)-MS2 interacting proteins. White dots are proteins that do not bind to neither Xist(F)-MS2 nor to MS2 alone; light grey dots are proteins defined as Xist(F)-MS2. See also experimental procedures for data normalization. RLU are Relative Light Units.

(D) Scatter-plot showing Xist(C)-MS2 interaction intensities (average from two biological replicates). The vertical dotted line delineates a threshold set at 3 normalized RLU to define Xist(C)-MS2 interacting proteins. White dots are proteins that do not bind to neither Xist(C)-MS2 nor to MS2 alone; black dots are proteins defined as Xist(C)-MS2. RLU are Relative Light Units.

FIG. 5: Functional assays reveal Xist-interacting proteins required for XCI in vivo

(A) Representative RNA FISH images of Xist-induced cells upon depletion of the indicated factors. Xist is shown in red and the X-linked gene Lamp2 in green. The dashed line delineates cell nuclei. Asterisks indicate Lamp2 expression from the active X chromosome. Arrowheads indicate Lamp2 expression from the inactive X chromosome that escapes XCI.

(B) Quantification of cells with bi-allelic Lamp2 expression assessed with RNA FISH, expressed as fold ratio over RLuc control. Data from three independent experiments are represented as mean±SD. Student's t-tests: *p<0.05; not significant (ns).

(C) RNA immunoprecipitation (RIP) of the HA-tagged RBM6 protein. Left panel, Western blot for RMB6. Right panel, RNA levels of the indicated transcripts in the input and in the immunoprecipitated eluates. All enrichments are normalized to GAPDH mRNA and to the input sample. Each RIP experiment was performed on two independent biological replicates. Data are presented as mean±SD; unpaired t-tests: **P<0.01; *P<0.05.

(D) RNA immunoprecipitation (RIP) of the HA-tagged ZZZ3 protein. Left panel, Western blot for ZZZ3. Right panel, RNA levels of the indicated transcripts in the input and in the immunoprecipitated eluates. All enrichments are normalized to GAPDH mRNA and to the input sample as described in experimental procedures. Each RIP experiment was performed twice on independent biological replicates. Data are presented as mean±SD; unpaired t-tests: **P<0.01; *P<0.05.

FIG. 6: Common Xist-binding proteins identified by incPRINT for the three tested Xist regions and in previous studies for the full-length Xist transcript

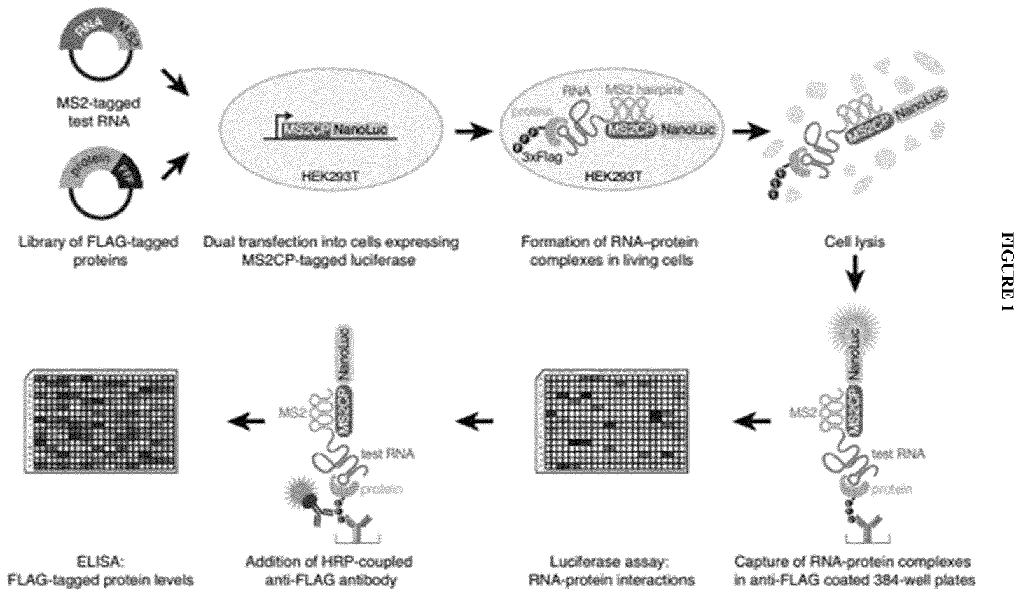

FIG. 7: incPRINT modifications to adjust it for a discovery of small molecule inhibitors targeting RNA-protein interactions. Cells will be transfected as described in FIG. 1 in a 96-well plate setup. Small molecule inhibitors are added to the cells after transfection. Cell viability is tested prior cell lyses.

FIG. 8: incPRINT modifications to adjust the technology to simultaneous testing of two RNAs of interest. A new stable HEK293 cell line expressing two luciferase detectors expresses Nluc and Fluc fused to MS2 coat protein and PP7 coat protein, respectively. incPRINT expression constructs are transfected into the stable HEK293 line as in the original incPRINT protocol.

FIG. 9: incPRINT modifications to adapt the technology for identification of RNA-protein interactions that depend on m⁶A RNA modification. The HEK293 cell line stably expressing MS2CP-Luc used in the core incPRINT experiment will be genetically modified to generate METTL3 and METTL14 null alleles resulting in loss of m⁶A RNA modification.

### EXAMPLE

An ideal proof-of-principle molecule to establish such a method is the long noncoding RNA Xist (X-inactive-specific transcript) given its vital role in mammalian X-chromosome inactivation (XCI) (Borsani et al., 1991; Brown et al., 1991), a previously identified set of functional protein interacting partners and the availability of a cellular read-out to test the functionality of the Xist-protein interactions (Chu et al., 2015b; da Rocha et al., 2014; Hasegawa et al., 2010; Jeon and Lee, 2011; McHugh et al., 2015; Minajigi et al., 2015a; Sarma et al., 2014; Zhao et al., 2008a). Xist is transcribed from the future inactive X chromosome, spreads in cis and underpins the gradual response resulting in transcriptional silencing of most X-linked genes (da Rocha and Heard, 2017; Moindrot and Brockdorff, 2016; Pinheiro and Heard, 2017). Similar to other regulatory lncRNAs, the ˜17-kb long Xist transcript contains several conserved sequence regions (called repeats A through F) that carry out distinct functions during the XCI process including initiation of gene silencing (A repeat), maintenance of the X-inactive state (F- and B-repeats) and proper chromosomal localization and focal accumulation of Xist (the C- and E-repeat) (Almeida et al., 2017; Beletskii et al., 2001; da Rocha et al., 2014; Nesterova et al., 2001; Ridings-Figueroa et al., 2017; Sarma et al., 2010; Senner et al., 2011; Sunwoo et al., 2017; Wutz et al., 2002). Extensive Xist-protein interactome analyses together with genetic screens and earlier candidate approaches have shown that these conserved Xist regions are required to interact with specific proteins at different steps of XCI (Chu et al., 2015b; Hasegawa et al., 2010; Jeon and Lee, 2011; McHugh et al., 2015; Minajigi et al., 2015b; Moindrot et al., 2015; Monfort et al., 2015; Sarma et al., 2014; Zhao et al., 2008b).

Experimental Procedures

Cell Culture

HEK293T Cells

To generate the stable monoclonal 293T cell line expressing NanoLuc-MS2CP fusion protein, the NanoLuc-MS2CP expression vector was transfected using polyethylemine (PEI). The cells were maintained in DMEM (Glbco 12007559) containing 10% fetal bovine serum (Gibco 11573397) and 1% penicillin/streptomycin (Gibco 15140122).

Mouse ES Cells

Female mouse G6pd-Fluo (clone E8_6) ES cells were derived from the TX1072 ES cell line (Schulz et al., 2014). The hybrid TX1072 cell line harbors a doxycycline-responsive promoter controlling Xist expression on the B6 X chromosome, and was derived from a cross of a TX/TXR26^{rtTA/rtTA} female (Savarese et al., 2006) with a male Mus musculus castaneus. The G6pd-Fluo cell line was engineered to harbor a GFP fluorescent reporter in the B6 allele and a tdTomato reporter in the Cast allele of the G6pd locus. The fluorescent proteins were inserted in frame with the G6pd protein-coding sequence, before the stop codon, and separated by a self-cleavable P2A peptide. This results in a single mRNA containing both G6pd and the fluorescent protein sequences, but two separate proteins due to P2A-mediated cleavage during translation (Kim et al., 2011). The fluorescent proteins contained a NLS sequence, to ensure their homogeneous localization in the nucleus. Cells were transfected using the Nucleofection™ technology (Lonza), simultaneously with the targeting vectors for both fluorescent proteins, each containing its respective resistance marker (pBR322-G6pd-GFP-Hygro and pBR322-tdTomato-Blast), and a pX459 containing a gRNA sequence targeting the G6pd protein coding sequence, for CRISPR/Cas9-mediated double-strand break at the integration site to improve homologous recombination efficiency.

24 hours post-nucleofection, the cells were selected for integration with consecutive pulses of 250 μg/mL hygromycin and 5 μg/mL blasticidin selection, until colonies were visible. Clones were genotyped for integration at the correct genomic location by PCR and DNA sequencing, to ensure no deletions or mutations in the protein-coding sequence. We detected a deletion downstream the 3′ homology arm in the Cast allele, containing the tdTomato integration. However, this deletion did not affect expression of neither G6pd nor tdTomato.

ES cells were cultured in high-glucose DMEM (Sigma) supplemented with 15% fetal calf serum (Eurobio, S59341-1307), 0,1 mM β-mercaptoethanol, 1000 U/mL leukemia inhibitory factor (LIF, Chemicon), and 2i (3 μM GSK3 inhibitor CT-99021, and 1 μM MEK inhibitor PD0325901). Xist expression was induced with Doxycycline (Sigma) for 48 hours before the cells were harvested for FACS analysis, or 24 hours for RNA FISH analysis.

esiRNA Transfections

The G6pd-Fluo ES cells were transfected with 0.1 ng esiRNA per cell (Kittler et al., 2007) with Lipofectamine 2000 Transfection Reagent (ThermoFisher Scientific), in Optimem I reduced serum medium (Thermo Ficher Scientific). esiRNAs (endoribonuclease-prepared short interfering RNAs) are produced by endoribonuclease-mediated digestion of in vitro transcribed dsRNAs spanning a gene-specific cDNA region of 300 to 600 bp. The digestion results in a pool of approximately 150 to 300 siRNA sequences that targets a specific mRNA and reduces off-target effects (Kittler et al., 2007).

Transfections were performed in 96-well plates using esiRNAs (Eupheria Biotech) for 37 different candidate genes listed in FIG. 5D and for luciferase and Spen, as negative and positive controls respectively. Doxycycline-mediated Xist expression was initiated 48 hours post-transfection, and cells were harvested for flow cytometry 48 hours post-doxycycline induction of Xist, to ensure that GFP protein levels had been properly depleted in luciferase esiRNA negative control samples, upon Xist-mediated G6pd silencing. For RNA FISH analysis, cells were harvested after 24 hours of Doxycycline induction of Xist expression.

To assess esiRNA knock-down efficiencies, 50 ng of total RNA were reverse-transcribed using the SuperScript IV kit (Invitrogen) followed by qPCR using SYBRgreen (Applied biosystems). Arppo mRNA was used to normalize RNA levels between samples.

Generation of Expression Constructs

NanoLuc-MS2CP Expression Vector

NanoLuc luciferase was amplified from the pNL1.1 plasmid (Promega) and cloned into the pCi-MS2 vector (Chao et al., 2007) using PstI and BamHI restriction sites. A Puromycin resistance gene was added to the plasmid using PvtI restriction site. To remove FLAG tag present in the original plasmid, a stop codon between MS2CP and FLAG tag was introduced by site-directed mutagenesis (Agilent).

RNA-10xMS2 Constructs:

The pCDNA3.1 plasmid (ThermoFisher) was modified to generate customized RNA-MS2 constructs. First, additional restriction sites (BstBI, AgeI, ClaI, AscI, PacI, BglII and SrfI) were incorporated between KpnI and BamH1 sites. Second, MS2 stem loops were inserted between BamHI and BglII sites. Xist(A), (F) and (C) fragments were PCR-amplified from a BAC 399K20 (covering chrX:100,578,985-100,773,006, mm9 genome assembly), and cloned upstream of the MS2 stem loops by Gibson assembly (New England Biolabs).

G6pd-Fluo Targeting Constructs:

G6pd-Fluo cells were generated by homologous recombination using pBR322-G6pd-GFP-Hygro and pBR322-G6pd-tdTomato-Blast as targeting vectors. Cloning was performed by Gibson assembly® (New England Biolabs) of a pBR322 plasmid (New England Biolabs) linearized with EcoRV and NruI, 500 bp left and right homology arms sequences that were PCR amplified from TX1072 genomic DNA, and the synthesized insert containing P2A peptide-Fluorescent protein-Resistance marker cassette (Biomatik). The gRNA sequence was inserted in the pX459 plasmid, linearized with BbsI (Ran et al., 2013).

To avoid CRISPR/Cas9-mediated double-strand break of the targeting vector upon cell transfection, the gRNA target sequence was mutagenized with silent mutations in the targeting vector, using the QuickChange II site-directed mutagenesis kit (Stratagene). The specific mutations were confirmed by plasmid DNA sequencing of the entire insert, and Cas9 activity was tested in both wild-type and mutagenized plasmids, using in vitro Cas9 assays with recombinant Cas9 (New England Biolabs) and in vitro transcribed gRNA using the MEGAshortscript™ kit (Ambion, Thermo Fischer).

Expression Clone Library Preparation

The transcription factor collection has been previously described (Taipale et al., 2012). The collection of RBPs and chromatin-associated proteins were cloned with Gateway recombination from the human ORFeome 5.1 (http://horfdb.dfci.harvard.edu/hv5/index.php) into a mammalian expression vector with a C-terminal 3xFLAG-V5 tag. The expression clones were verified by restriction enzyme digestion.

The incPRINT Method

Plate Coating and Blocking

384-well plates (Greiner Bio-One 781074) were coated with the anti-FLAG M2 antibody (Sigma). Following an overnight incubation in the antibody dilution (10 μg/ml, in 1X PBS), plates were blocked for an hour at room temperature in 1% BSA, 5% sucrose, 0.5% Tween 20 in 1X PBS.

Cells Transfections

Plasmids encoding the RNA-MS2 of interest and the 3xFLAG-tagged test proteins were co-transfected into a 293T stable cell line expressing the NanoLuc luciferase fused to MS2CP. The day before transfection, cells were seeded in 96-well plates (30.000 cells per well). Co-transfections were performed using polyethylemine (PEI) (150 ng of each plasmid per well).

Pull-Downs and Luminescence Measurement

Two days after transfection, cells were washed twice with 1X PBS and lysed in ice-cold RQ1-HENG buffer (20 mM HEPES-KOH [pH7.9], 150 mM NaCl, 10 mM MgCl2, 1 mM CaCl2, 5% glycerol, 1% Triton X-100, supplemented with protease inhibitors) containing 30 U/ml of RQ1 DNAse (Promega M6101). After lysis (10 minutes, 4° C.) and DNAse incubation (30 minutes, 37° C.), the lysates were transferred to 384-well plates coated with anti-FLAG M2 antibody. Following a three hours-incubation at 4° C., plates were washed six times with RQ1-HENG buffer. Furimazine substrates (a gift from Y. Jacob, Institut Pasteur; Promega) were then added to the plates and luminescence in each well was measured with a plate reader (Biotek Synergy Neo). Following luminescence measurement, HRP-conjugated anti-FLAG antibody (Abcam) in ELISA buffer (1X PBS, 1% goat serum, 1% Tween 20) was added to each well. After 1 hour of incubation at room temperature, plates were washed with 1X PBS, 0.05% Tween 20, HRP substrates (Pierce) were added and ELISA signals were detected using a plate reader (see above).

All 96- and 384-plate washes were performed with an automated plate washer (Biotek EL406).

incPRINT Data Analysis and Normalization

For each studied RNA (e.i. MS2, Xist(A)-MS2, Xist(F)-MS2, Xist(C)-MS2), all 3xFLAG-tagged proteins were tested in duplicate with independent transfections. After measuring the Nanoluciferase and ELISA luminescence, log₂-transformed ELISA values were binned to assess the distribution of the 3xFLAG-tagged proteins expression. The population of proteins showing the lowest ELISA signals were filtered-out. Interaction values between a test RNA-MS2 and a test protein were defined as the average between the two Luciferase luminescence replicates, without taking protein expression into account. To ensure robustness of the interaction analysis, Luciferase replicates that showed the highest discrepancy (average/SD>1.5, unless both duplicates showed a high interaction score) were removed from the data set.

To compare the interaction intensities among individual RNA-MS2 transcripts (FIG. 3A; 4B-E), raw luminescence intensities were normalized. A set of proteins exhibiting a luminescence signal with MS2 alone was defined as “common binders”, assumed to interact with all tested RNA-MS2 transcripts. For each tested RNA-MS2 transcript, the median interaction score of this set of “common binders” was calculated and used to normalize all raw luminescence intensities measured for the corresponding RNA-MS2.

The comparison between Xist(A)-MS2, Xist(F)-MS2 and Xist(C)-MS2 interactors (FIG. 4C), includes all test proteins showing interaction with at least one RNA, and expressed in all three assays.

GFP Reporter-Based XCI Assay

48 hours post-transfection, Xist expression was induced in undifferentiated ES cell lines by addition of 1 μg/mL of Doxycycline to the culture medium. 48 hours after Xist induction, cells were harvested using TrypLE Express Enzyme (Thermo Fisher Scientific), and resuspended in 1X PBS. For flow cytometry analysis, the Cytoflex analyser (Beckman) was used to measure GFP and tdTomato expression that served as reporters for X-linked gene expression/silencing. Analysis of the flow cytometry data was performed with FlowJo® and the NovoExpress software (ACEA Biosciences).

Xist-mediated repression efficiency was defined as a decrease in the percentage of GFP-positive cells in the tdTomato-positive G6pd-Fluo cell population upon Xist Doxycycline induction. This decrease was calculated for each tested condition and normalized by the median repression efficiency of the 37 tested conditions. Relative repression was calculated as average (n>4 for each tested condition).

RNA FISH

RNA FISH was performed as previously described (Chaumeil et al., 2008). 48 hours post-transfection, Xist expression was induced in undifferentiated ES cell lines by addition of 1 μg/mL of Doxycycline to the culture medium. 24 hours upon Xist induction, cells were harvested using TrypLE Express Enzyme (Thermo Fisher Scientific), washed with 1X PBS, and adsorbed onto Poly-L-Lysine (Sigma)-coated glass coverslips during 10 minutes. Fixation was performed with 3% para-formaldehyde for 10 minutes at room temperature, followed by permeabilization in 1X PBS containing 0.5% of Triton X-100 and 2 mM of Vanadyl-Ribonucleoside complex (New England Biolabs), for 5 minutes at 4° C. Coverslips were washed 3 times in 70% ethanol, and preserved at −20° C. in 70% ethanol. Prior to hybridization, coverslips were dehydrated with increasing concentrations of ethanol (80%, 95%, 100% twice, 5 minutes each), and air-dried. Transcription of the X-linked gene Lamp2 was detected with a BAC spanning its genomic region (RP24-173A8). The Lamp2 probe was labelled with dUTP-Spectrum Green (Enzo, Life Sciences) by nick translation (Abbot). The probes were precipitated with ethanol, resuspended in formamide at 37° C., denatured at 75° C. for 10 min, and competed with mouse Cotl DNA (Thermo Fisher) for 1-2 hours at 37° C. Xist was detected with a dUTP-Spectrum Red (Enzo, Life Sciences) nick translation probe from a plasmid spanning its genomic region (Chaumeil et al., 2008). The Xist probe was prepared as described above for the Lamp2 probe, except for the competition step, that was not performed. Probes were mixed and co-hybridized in FISH hybridization buffer (50% formamide, 20% dextran sulfate, 2x SSC, 1 μg/μL BSA (New England Biolabs), 10 mM Vanadyl-ribonucleoside) overnight at 37° C. Coverslips were washed 3×6 minutes in 50% formamide in 2x SSC, pH7.2, at 42° C., followed by 2×5 minutes washes in 2x SSC at 42° C. Nuclei were counterstained with 0.2 mg/mL of 4′,6-Diamidine-2′-phenylindole dihydrochloride (DAPI) in 2x SSC during 3 minutes at room temperature, and mounted onto glass slides using VectaShield mounting medium. Images were acquired using the wide-field DeltaVision Core microscope (Applied Precision) and the inverted confocal Spinning Disk Roper/Nikon-FRAP microscope. 3D image stacks were analyzed with ImageJ.

Western Blot Analysis

To assess knockdown efficiency at the protein level, G6pd-Fluo cells were transfected with 2.5 of esiRNAs for RLuc (as a negative control) and the candidate factors Rbm6 and Zzz3. Whole cell extracts were prepared with RIPA buffer. Western blot analysis was performed with antibodies detecting RBM6, ZZZ3 and Tubulin (CP06 mouse monoclonal antibody (DM1A), Merck Millipore).

RNA Immunoprecipitation (RIP)

RIP experiments were performed using corresponding HA-tagged RBM6 and ZZZ3 G6pd-Fluo cell lines. ˜7 million cells were treated with Doxycycline (1 ug/ml) for 16 h. Cells were washed once with ice-cold 1X PBS and UV-crosslinked using 800 mJ/cm2, at 254 nm (Stratalinker, Stratagene). Cells were then collected, pelleted and lysed 10 min on ice in 200 ul of RIPA buffer containing protease inhibitors (Roche) and RNAsin (Promega). Cell lysates were sonicated using a Bioruptor (Diagenode) (6 cycles; 15 sec on, 30 sec off), and centrifuged for 10 min at 15,000 g. Supernatant were collected and diluted in RQ1-HENG buffer (to 1.2 ml). 50 ul of anti-HA beads slurry (Pierce, 88836) was washed twice with 1 ml of RQ1-HENG buffer and incubated with the cell lysate 2 h at 4° C. Following the incubation, beads were washed twice with TNM600 buffer (50 mM Tris-HCl [pH7.8], 0.6 M NaCl, 1.5 mM MgCl2, 0.1% NP40), and twice with TNM100 buffer (50 mM Tris-HCl [pH7.8], 0.1 M NaCl, 1.5 mM MgCl2, 0.1% NP40). Elution was performed in 50 ul Laemmli buffer (2 min, 90° C.), RNA was extracted (Trizol-Chloroform) and treated with TURBO DNase (Ambion). 50 ng of RNA were reverse-transcribed using the SuperScript IV kit (Invitrogen) followed by qPCR using SYBRgreen (Applied biosystems). Gapdh mRNA was used to normalize RNA levels between samples.

Protocol 1

Consumables and Reagents:
Lumitrac 600 white high-binding 384-well microplates (Greiner Bio-One, 781074)
Costar 96-well flat bottom tissue culture plates (Fisher, 07-200-92)
Mouse anti-FLAG M2 (Sigma, F1804)
DDDK tag antibody (goat), HRP-conjugated (Abeam, ab1238)
SuperSignal ELISA Pico Chemiluminescent Substrate, 250 ml (Pierce 37069)
Polyethylenimine “MAX” (Polysciences, 24765)
OptiMEM serum reduced media (Invitrogen, 22600-134)
Furimazine (Promega, N1120)

Buffers:
anti-FLAG coating solution (plate coating part I) 10 ug/ml anti-FLAG M2 (Sigma, F1804-5MG) in PBS1X (1:200 dilution)
Blocking buffer (plate coating part II): 1% BSA; 5% sucrose; 0.5% Tween20; 1X PBS
PBS-Tween (ELISA washes): 0.05% Tween20; 1X PBS
RQ1-HENG buffer (=Lysis-IP buffer, used for lysis, incubation and washes): HEPES KOH pH7.9 (20 mM); NaCl (150 mM); MgCl2 (10 mM); CaCl2 (1 mM); Triton X100 1%; Glycerol 5%

For lysis, DNAse treatment and incubation add:

RQ1 DNAse (0.03 units/ul)

Aprotinin, leupeptin, pepstatin (1 ug/ml each) and PMSF (0.5 mM)
Luciferase assay buffer: Tris-HCl pH7.5 (20 mM); EDTA (1 mM); KCl (150 mM); Tergitol NP9 (0.5%) (before reading, Add Furimazine substrate: 1/200 dilution in buffer)
ELISA buffer: 1% Tween20; 1% goat serum; 1X PBS
RNAse buffer: Tris-HCl pH8.0 (50 mM); EDTA (10 mM); RNAse A (Qiagen 19101) (100 ug/ml)

incPRINT Step-by-Step Protocol:
Library: 36×96-well plates of FLAG-tagged proteins
The protocol is designed for one test RNA done in duplicates

Coating and Blocking of the Plates

Coat 384-well plates in advance (can be stored at 4° C. and used within a month)
1. Prepare anti-FLAG M2 solution in 1xPBS
2. Add 20 μl/well antibody solution to 384-well plates (use plate filler)
3. Cover plates and incubate overnight on a shaking platform (room temp)
4. Invert the plates to remove the antibody solution
5. Add 80 μl blocking buffer per well (use plate filler)
6. Incubate plates with blocking buffer for at least 1 hour at room temperature
7. Invert the plates to remove the blocking buffer
8. Use immediately, or alternatively cover plates with sealing tape and store at 4° C.

Transfection of Cells

Day 0:
1. Seed HEK293 cells expressing luciferase detector into 96-well plate such that they are ˜95% confluent on the day of transfection (30,000 cells/well) (use plate filler)
2. On a separate V-bottom 96-well plate, prepare the DNA transfection plate(s).
Each well contains:
   150 ng of the pcDNA3.1-RNA-10xMS2 plasmid
   150 ng of the pcDNA3.1-FLAG-tagged protein plasmid
(Prepare all mixes to transfect in duplicates)
Store the DNA transfection plate(s) at −20° C.

Day 1:
3. Add 100 μl of a mix containing OptiMEM and PEI (1,2% PEI) to each well with DNA (=Volume for a duplicate)
4. Incubate 30 min at room temperature
5. Carefully add transfection mix to wells (50 ul/well), to avoid losing cells
6. 48 hours after transfection, proceed to the incPRINT assay

incPRINT Assay

Day 3:
1. Wash cells twice with 1xPBS (100 μl/well) (use plate washer)
2. Lyse cells with ice-cold RQ1-HENG buffer*(80 μl/well) (*the buffer contains RQ1 DNAse and protease inhibitors that are added fresh before use)
3. Incubate 10 min, shaking, in cold room (cell lysis)
4. Incubate 30 min, at 37° C. (DNAse treatment)
5. Transfer 60 μl of lysate to anti-FLAG coated 384-well plates (Optional: keep the rest -approx. 20 ul- at 4° C., if input needed)
6. Incubate plates for 3 hours at 4° C. on a rocking or shaking platform (Do not cover plates with a sealing film)
7. Wash plates 6 times with RQ1-HENG buffer (100 μl/well) (use plate washer)
8. After the last wash, remove any remaining liquid
9. Add 20 ml luciferase assay buffer with furimazine (use plate filler)
10. Measure luciferase luminescence with a plate luminometer
11. Remove the reagent
(Steps 12-15: RNAse elution. Optional. If not required, go to step 15)
12. Add 50 ul of RNAse buffer to each well
13. Incubate 30 min at 37° C.
14. Wash plate 3 times with RQ1-HENG buffer (100 μl/well) (use plate washer)
15. Measure luciferase luminescence with a plate luminometer
16. Add 30 μl/well of ELISA detection antibody in ELISA buffer (use a distinct plate filler, or the injector of the luminometer, to avoid contamination)
17. Incubate plates (no cover) for 1 h at room temperature on a rocking platform
18. Wash plates 7 times with 1xPBS/Tween (use plate washer)
19. Remove the remaining liquid
20. Add 30 μl diluted ELISA substrate to each well (use plate filler)
21. Read HRP luminescence immediately with a plate luminometer

1. Results

incPRINT Reliably Detects Cellular RNA-Protein Interactions

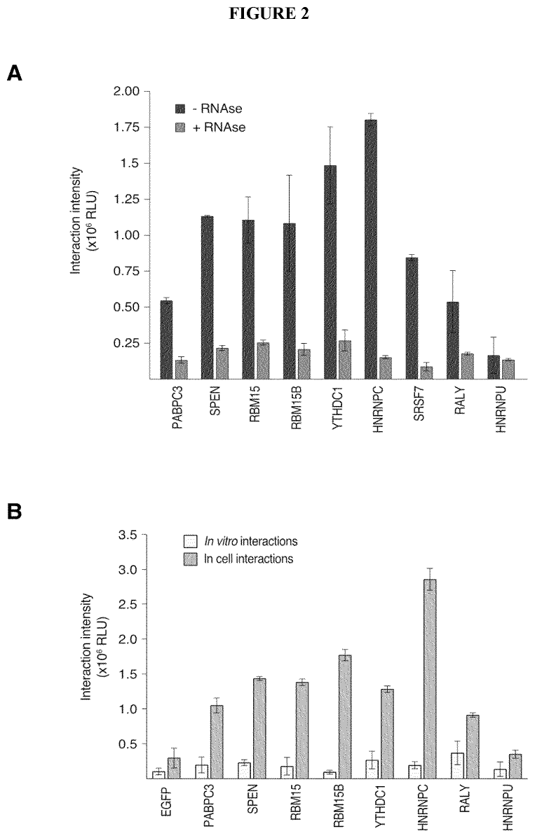

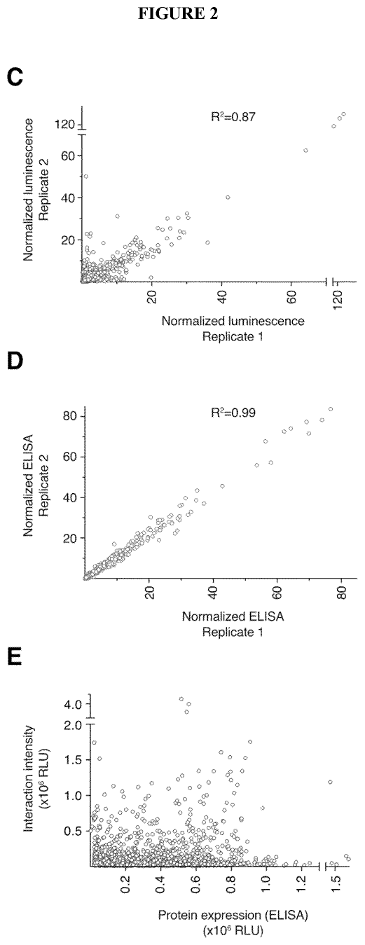

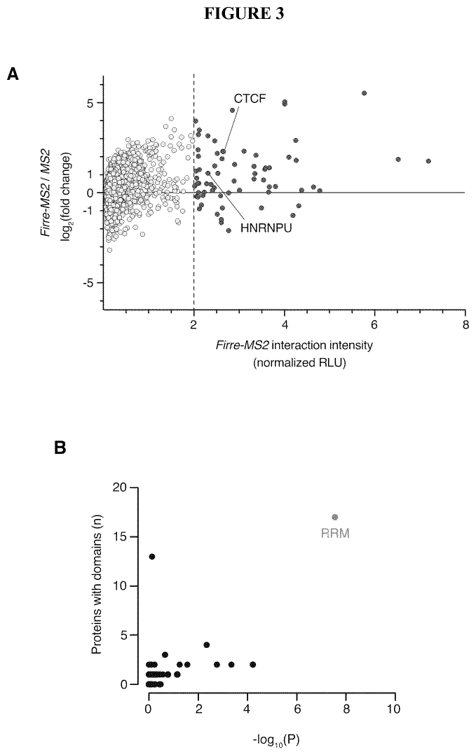

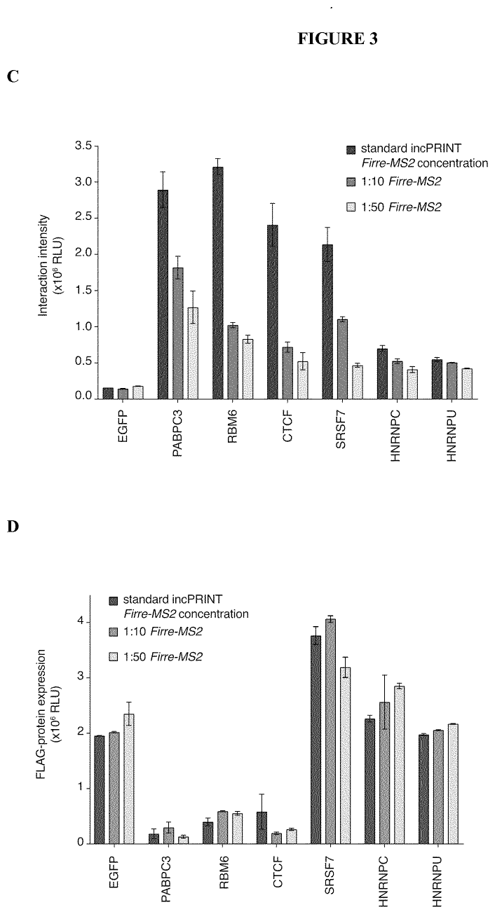

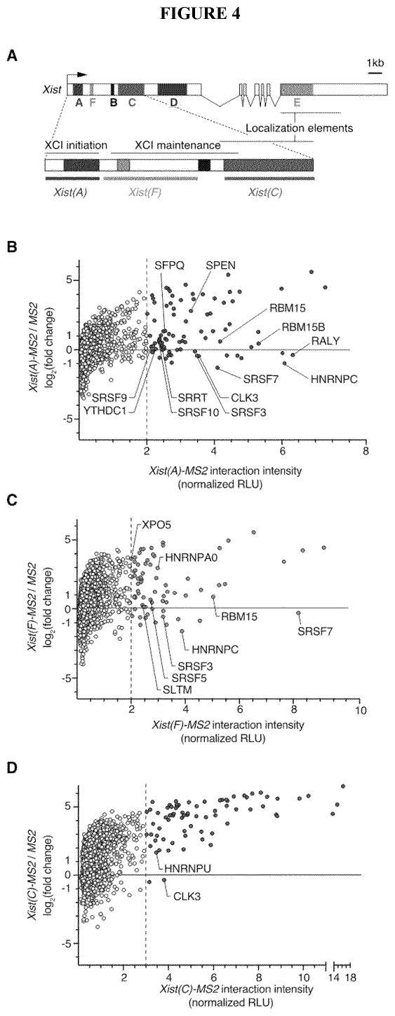

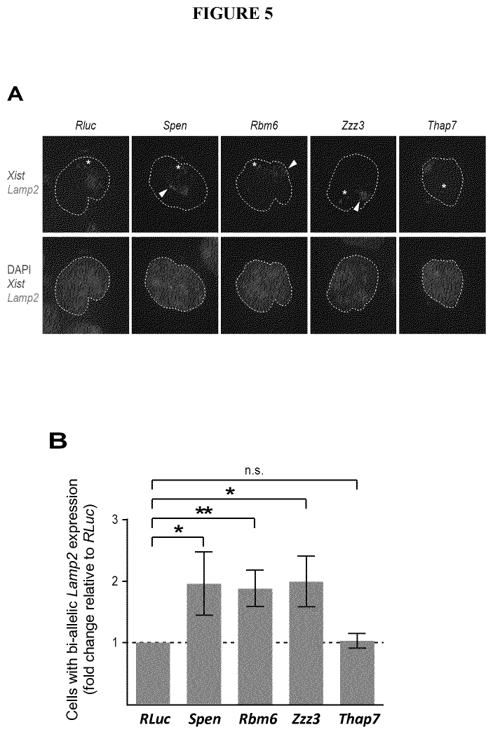

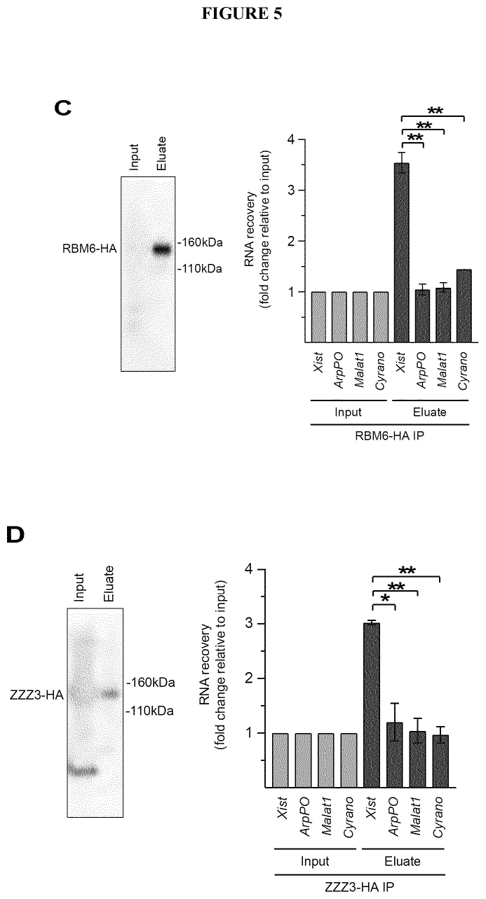

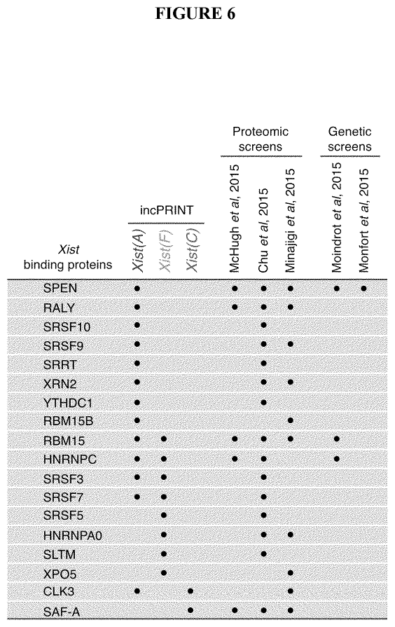

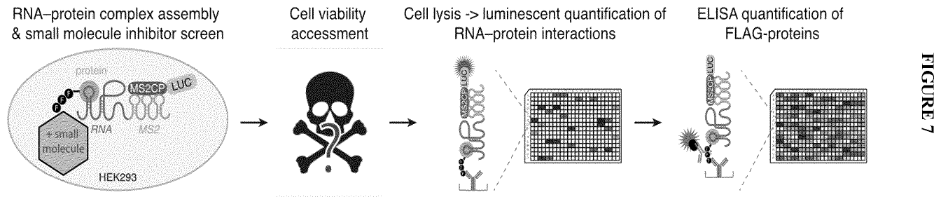

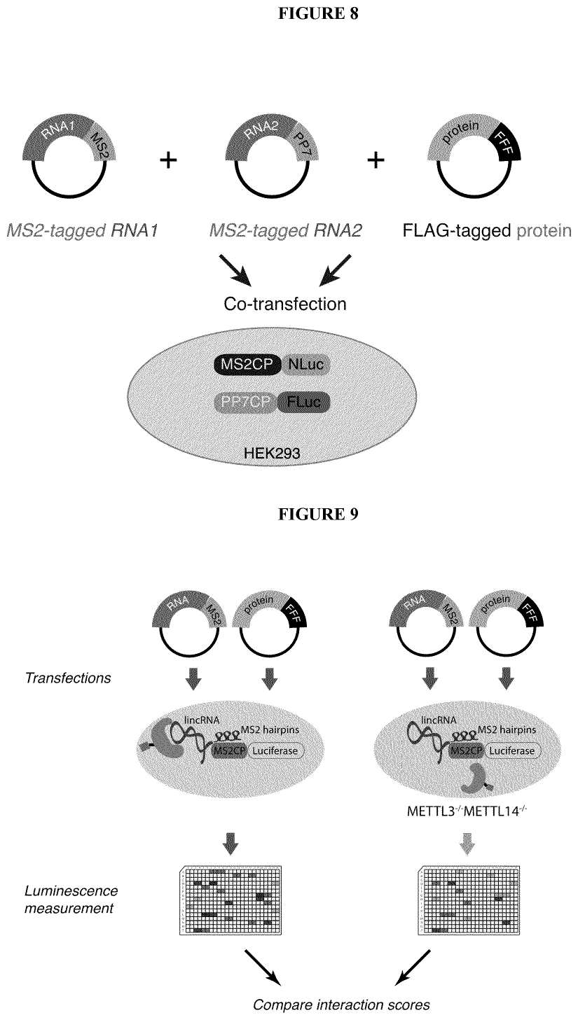

To establish the incPRINT method, the inventors performed a series of small-scale experiments using a ˜1-kb conserved region located at the 5′ end of the 17-kb Xist transcript called A-repeat hereafter referred to as Xist(A) (Nesterova et al., 2001). Because several Xist(A)-protein interactions have been well established (Chu et al., 2015b), they served as controls in our initial incPRINT experiments. We engineered a construct to express Xist(A)-MS2 and assayed its ability to interact with a selected set of previously identified Xist-binding proteins (Chu et al., 2015b; McHugh et al., 2015; Minajigi et al., 2015a) (FIG. 2A). The non-discriminatory poly(A)-binding protein PABPC3 was used to control for RNA expression. incPRINT luminescence detected specific interactions of Xist(A)-MS2 with SPEN, RBM15, RBM15b, YTHDC1, HNRNPC, SRSF7 and RALY, whereas HNRNPU showed basal binding (FIG. 2A). Importantly, while expression of test proteins detected by ELISA remained mostly unchanged, the RNA-protein interaction signal measured by luciferase was abolished after treatment with RNAase, demonstrating that the interactions between the tagged proteins and the luciferase detector were bridged by RNA (FIG. 2A,).

To optimize the number of MS2 stem loops used to tag the tested RNA, Xist(A) was fused with two, four, six, ten or 24 MS2 stem loops, and their interactions with a set of control proteins were tested in a small-scale incPRINT experiment. An increase in the luminescence intensity directly correlated with the increased number of MS2 stem loops up to ten stem loops with no marked increase in binding to the EGFP control (Data not shown). Therefore, in all subsequent incPRINT experiments, RNAs were tagged with ten MS2 stem loops.

To determine whether the RNA-protein interactions detected by incPRINT occurred in cell or in vitro due to the potential re-association of the RNA-protein complexes after cell lyses (Mili and Steitz, 2004; Riley et al., 2012; McHugh, Russell, and Guttman 2014), the luminescence signal from two independent experiments was measured. In the first experiment, Xist(A)-MS2 RNA and FLAG-tagged test proteins were co-transfected as described above. In the second experiment, Xist(A)-MS2 RNA and FLAG-tagged test proteins were transfected separately in two different cell populations and pooled together only after the cell lysis step permitting the formation of RNA-protein complexes exclusively in vitro (Data not shown). We found that interactions were preferentially detected in the standard incPRINT conditions when Xist(A)-MS2 RNA and the FLAG-tagged proteins were co-transfected (FIG. 2B). These results suggest that whenever an interaction signal was detected by incPRINT, it stemmed from the RNA-protein complexes formed in cell, whereas re-association of RNA-protein complexes appeared as a neglectable background (FIG. 2B). Taken together, these experiments establish that incPRINT measures cellular RNA-protein interactions using a chemiluminescence readout.

incPRINT Enables High-Throughput Detection of RNA-Protein Interactions

To test the scalability of incPRINT for systematic identification of RNA-protein interactions, the inventors generated a customized library of ˜3000 FLAG-tagged human proteins including the majority of all known RBPs (Baltz et al., 2012; Castello et al., 2012), transcription factors (Taipale et al., 2012) and chromatin-associated proteins. To strengthen the confidence of the incPRINT-identified RNA-protein interactions, all interactions were assayed in biological duplicates generating two luminescence (RNA-protein interaction intensity) and two ELISA (test protein expression level) values for each tested RNA-protein couple. We interrogated the protein library with Xist(A)-MS2. After filtering out the proteins expressed at insufficient levels (Experimental procedures), interaction data was analyzed for 2405 proteins. incPRINT's reproducibility was assessed by calculating correlation scores between the biological duplicates for both the luminescence (FIG. 2D; R²=0.87) and ELISA signals (FIG. 2E; R²=0.99). Importantly, no correlation between luminescence and ELISA values was detected, indicating that the interaction intensities were not a mere reflection of the protein expression levels (FIG. 2F). In summary, our data demonstrate that incPRINT is a scalable high-throughput method that reproducibly measures RNA-protein interactions in cell.

incPRINT Identifies Proteins Interacting with Lowly Expressed RNAs

Next, the inventors sought to test incPR1NT applicability for identification of proteins interacting with transcripts expressed at low endogenous levels. Identification of proteins associated with low copy number RNAs is particularly challenging due to the general low efficiency of RNA purifications and a large amount of material required for mass spectrometry when using AP-MS approaches. Because Firre is a functionally important lncRNA that modulates higher-order nuclear architecture across chromosomes (Hacisuleyman et al., 2014) of a rather low endogenous abundance of ˜20 molecules per cell (based on RNA-Seq data), the inventors decided to analyze Firre's RBP-interactome with incPRINT. The full-length Firre transcript tagged by MS2 was expressed ˜40-fold higher than endogenous Firre in HEK293 cells that were used for incPRINT (Data not shown). As reported for the endogenous transcript (Hacisuleyman et al., 2014), Firre-MS2 was preferentially localized to the nucleus (Data not shown). Results of the large-scale incPRINT experiment interrogating our library of 3000 proteins with Firre-MS2 are presented as Firre-MS2 interaction intensity plotted against the interaction score enrichment of Firre-MS2 over MS2 to report proteins with a non-exclusive Firre binding (FIG. 3A). Because incPRINT-tested transcripts have different expression levels (Data not shown), the interaction scores were normalized when comparing large-scale interaction data across different RNAs. Briefly, proteins interacting with MS2-alone RNA were defined as common binders of all tested MS2-tagged RNAs. Their median interaction score was calculated for each tested RNA and used to normalize raw luminescence intensities (see also Experimental Procedures).

incPRINT assaying Firre-MS2 and MS2 RNAs revealed that the majority of the proteins do not interact with either RNA (FIG. 3A, white dots), whereas a set of specific proteins was identified as Firre interactors (FIG. 3A, black dots). Among Firre-interacting proteins, a fraction of RBPs showed also binding to MS2. These proteins were not dismissed as Firre interaction partners in our data set, as their non-exclusive binding does not preclude their functional interaction with Firre RNA (FIG. 3A, black dots). Importantly, incPRINT identified both already known and novel Firre-interacting proteins (FIG. 3A). Both, CTCF and HNRNPU, identified by incPRINT as Firre interaction partners, have been previously validated as binding partners of Firre important for its function (Hacisuleyman et al., 2014) (FIG. 3A). ENCODE eCLIP data (reference) available for XX RBPs identified by incPRINT as Firre-interacting proteins confirmed Firre binding of XX proteins, validating thereby the incPRINT method (Data not shown). Consistent with the role of Firre in the nuclear organization (Hacisuleyman et al., 2014), a set of novel Firre interactors identified by incPRINT were chromatin-associated proteins including CHD1, POU5F1, JARID2, CTCF, EPC1, SATB1, MECP2 and AEBP2. Moreover, the protein domain analysis showed that Firre-interacting proteins identified by incPRINT were significantly enriched for the RNA recognition motif (RRM) (FIG. 3B). Together, it was demonstrated that incPRINT enables identification of proteins associated with transcripts expressed at low endogenous levels.

The inventors also further provided evidence that detection of RNA-protein interactions by incPRINT is not dependent on RNA overexpression (FIG. 3C-D). When testing a set of proteins with different concentrations of Firre-MS2, the specificity of RNA-protein interactions remained unaffected. Whereas RNA overexpression allowed better separation of the interactions from the background, the protein interactions with Firre-MS2 expressed at low levels (1:50) were robustly detectable. The sensitive luciferase detector tethered to the test RNA through the high affinity MS2-MS2CP interaction rather than RNA overexpression per se is a key component of the incPRINT system. Importantly, this signal was not associated with the test protein expression levels (FIG. 3D). Together, these results demonstrate the utility of incPRINT to identify proteins associated with transcripts expressed at low endogenous levels.

incPRINT Identifies RNA Region-Specific Interaction Partners

Because many lncRNAs function as modular scaffolds, enabling binding of specific RBPs to discrete RNA domains (Engreitz et al., 2016; Guttman and Rinn, 2012), the inventors tested if incPRINT allows the identification of RNA domain-specific interactions. An ideal proof-of-principle molecule is the lncRNAXist given its vital role in mammalian X-chromosome inactivation (XCI) (Borsani et al., 1991; Brown et al., 1991) and its modular structure and function. Similar to other regulatory lncRNAs, the ˜17-kb long Xist transcript contains several conserved sequence regions (called repeats A through F) that carry out distinct functions during the XCI process including initiation of gene silencing (A repeat), maintenance of the X-inactive state (F- and B-repeats) and proper chromosomal localization and focal accumulation of Xist (the C- and E-repeat) (Almeida et al., 2017; Beletskii et al., 2001; da Rocha et al., 2014; Nesterova et al., 2001; Ridings-Figueroa et al., 2017; Sarma et al., 2010; Senner et al., 2011; Sunwoo et al., 2017; Wutz et al., 2002) (FIG. 4A). Moreover, several independent studies have previously identified and validated a set of functional Xist-protein interactions (Chu et al., 2015b; da Rocha et al., 2014; Hasegawa et al., 2010; Jeon and Lee, 2011; McHugh et al., 2015; Minajigi et al., 2015a; Sarma et al., 2014; Zhao et al., 2008a).

The inventors applied incPRINT to three conserved regions of Xist, Xist(A), Xist(F) and Xist(C) (FIG. 4A). When expressed in HEK293 cells used for incPRINT, each individual Xist-MS2 fragment showed a different level of expression ranging from ˜60-fold increase for Xist(A) to the expression levels comparable to endogenous Xist for Xist(C) (Data not shown). All individual Xist-MS2 fragments were preferentially expressed in the nucleus suggesting their localization to the correct cellular compartment (Data not shown). Xist(A), Xist(F) and Xist(C) regions were interrogated with our library of ˜3000 proteins. For each tested Xist region, the incPRINT results are shown as RNA-MS2 interaction intensity plotted against the interaction score enrichment of RNA-MS2 over MS2 (FIG. 4B-D). To compare signals across individual Xist regions, the interaction scores for each Xist region and MS2 alone were normalized as described for the lncRNA Firre (see also Experimental Procedures). Analyzing incPRINT data, it was found that the majority of proteins did not bind to any of the tested Xist fragments (FIG. 4B-D, white dots), whereas specific sets of proteins were identified to interact with each individual Xist region (FIG. 4B-D, black dots). As expected, a fraction of proteins showed a non-exclusive binding to Xist and interacted also with MS2. Similar to Firre, these proteins were not dismissed as Xist interaction partners in our data sets as their binding to MS2 does not exclude their functional interaction with Xist. Importantly, among incPRINT-identified Xist-interacting proteins, the inventors found well-known interaction partners of Xist that have been identified in previous studies using the full-length transcript (Chu et al., 2015b; McHugh et al., 2015; Minajigi et al., 2015a) (highlighted in FIG. 4B-D, FIG. 6).

Next, the sets of incPRINT-identified proteins and their interaction scores were compared for each interrogated Xist region. We found that each individual Xist fragment interacted with a set of proteins specific to each region, with a very minor fraction of proteins interacting with all three Xist regions. As such, applying incPRINT to three conserved regions of Xist enabled identification of RNA region-specific RBPs. In addition, incPRINT allowed assignment of RBP binding to specific RNA regions previously determined to bind the full-length Xist transcript (Chu et al., 2015b; McHugh et al., 2015; Minajigi et al., 2015a). For instance, incPRINT identified SPEN as a Xist(A)-specific interactor confirming previous findings (Chu et al., 2015b; Lu et al., 2016). Similarly, RBM15, RBM15B and YTHDC1 were identified by incPRINT to interact specifically with the Xist(A)- and Xist(F)- but not Xist(C)-regions (FIG. 4C) confirming their reported binding to the 5′ end of Xist (Patil et al., 2016). Moreover, the inventors identified an Xist(C)-specific interaction of the SAF-A protein (also known as HNRPU) previously shown to be involved in Xist localization (Chu et al., 2015b; Hasegawa et al., 2010; McHugh et al., 2015) (FIG. 4C, FIG. 6).

Consistent with the differential functions of the tested Xist regions, Gene Ontology term enrichment showed a functional difference between the protein interactomes of the three Xist regions confirming specificity of the RNA-protein interactions identified by incPRINT. While A- and F-associated proteins were enriched for RBPs involved in RNA processing, the C-repeat region preferentially interacted with DNA-binding proteins involved in transcriptional regulation (Data not shown). Consistent with our GO analysis and the reported differential functions of the Xist regions, the RBP domain analysis demonstrated that Xist(A)-interacting proteins were enriched for the SPOC (Spen paralog and ortholog C-terminal) and RRM protein domains, Xist(F)-interacting proteins were enriched for the RRM domains and Xist(C)-interacting proteins showed no enrichment, further highlighting specificity of incPRINT-identified protein sets for each Xist region. In summary, the incPRINT method successfully retrieved known Xist-protein interactions and uncovered novel RBPs. By identifying specific sets of proteins interacting with individual conserved regions of a modular lncRNA, the inventors demonstrated that using incPRINT on distinct regions of RNA transcripts enables the identification of region-specific RNA-protein interactions.

Assessment of incPRINT-Identified Xist Interacting Proteins Involved in Gene Silencing During XCI

Because Xist has a well-characterized cellular function in gene silencing during XCI, the inventors set out to examine the physiological relevance of some of the novel Xist-protein interactions identified by incPRINT. To track the dynamics of X-linked gene silencing, the inventors modified the previously described polymorphic TX1072 cell line that resulted from a cross between Mus musculus domesticus (B6) and Mus musculus castaneus (Cast) mouse strains and enabled doxycycline-induced Xist expression from the endogenous B6 Xist locus, hence triggering XCI in undifferentiated ESCs (Schulz et al., 2014). Insertion of a GFP reporter gene into the B6 G6pd locus and a tdTomato reporter gene into the Cast G6pd allele of the TX1072 cell line (hereafter referred to as G6pd-Fluo cell line) enabled fluorescent monitoring of the expression of the X-linked gene G6pd that is normally silenced during the early stages of XCI (Borensztein et al., 2017; Patrat et al., 2009) (Data not shown). In undifferentiated G6pd-Fluo ES cells that do not express Xist, both G6pd alleles were expressed and detectable by FACS (Data not shown). Upon doxycycline induction of Xist, silencing of the X^{B6} chromosome could be tracked by decreased GFP expression, whereas expression of tdTomato on the active X^{Cast} chromosome was unaltered (Data not shown). The effectiveness of XCI was assessed after depletion of 37 incPRINT-identified Xist-binding proteins using multi-well FACS analyses, whereas SPEN depletion served as a control known to reduce Xist-mediated gene silencing of X-linked genes (Chu et al., 2015b; McHugh et al., 2015; Moindrot et al., 2015; Monfort et al., 2015) (Data not shown). The 37 candidates for functional analyses were selected based on their high Xist-interaction score defined by incPRINT and their novel association with Xist, in particular with the A-repeat region. SPEN depletion led to reduced G6pd gene silencing upon XCI, resulting in an increase in GFP expression (Data not shown). Similar to SPEN, depletion of several other candidate proteins including ZZZ3, CWC22, CUL2, RBM6 consistently resulted in similarly defective XCI initiation (Data not shown). Reduced G6pd-GFP silencing was consistently observed upon depletion of the novel Xist-associated RBPs, although the impact on XCI was partial, likely due to incomplete doxycycline induction of Xist (Wutz et al., 2002) and the knockdown efficiency of the individual tested proteins (Data not shown).

The inventors further confirmed the role of RBM6 and ZZZ3 in the initiation of XCI by single cell RNA fluorescent in situ hybridization (RNA FISH) assessing expression of endogenous Lamp2, another X-linked gene that is normally silenced during XCI initiation (Patrat et al., 2009). The effect of Rbm6 and Zzz3 depletion was tested by analysis of Xist and Lamp2 expression whereas depletion of Spen and Thap7 served as positive and negative controls, respectively (FIG. 5A, B). Consistent with our FACS analyses, depletion of Rbm6, Zzz3 and control Spen led to reduced silencing of Lamp2, whereas its XCI-induced mono-allelic expression remained unaltered upon control Thap7 depletion (FIGS. 5A and 5B). Importantly, these defects in X chromosome silencing were not triggered by altered expression of Xist/Tsix upon depletion of the individual proteins (Data not shown). Moreover, to confirm Xist interaction with RBM6 and ZZZ3 in vivo, the inventors tested if Xist co-precipitates with both proteins in Dox-induced G6pd-Fluo ES cells. Both proteins were HA-tagged in G6pd-Fluo ES cells (see Experimental Procedures for details) and RNA immunoprecipitation (RIP) analyses were performed after Dox-induction of Xist. RIP qRT-PCR identified a significant enrichment of the Xist transcript with RBM6 and ZZZ3 proteins, confirming their interaction in vivo (FIG. 5C, D). In summary, it was demonstrated that there are novel, functionally important interactors among incPRINT-identified proteins.

2. Results Conclusions

Several key features of incPRINT distinguish it from other currently employed RNA-centric methods for identification of RNA-protein interaction and make it suitable for various custom applications. First, incPRINT does not rely on RNA purification, which has generally low efficiency and requires large amounts of material. By ectopically expressing both the test RNA and protein components, incPRINT is also not limited by RNA low copy number and can be applied to any RNA of interest. Second, some of the incPRINT-identified RNA-protein interactions might represent transient yet functional RNPs. Third, the ability of incPRINT to measure cellular RNA-protein interactions one-by-one, independently of the cell physiological state is particularly relevant for defining RNA-bound proteomes of transcripts that display a dynamic composition during development and/or cellular differentiation, as observed with Xist throughout different stages of XCI (Chu et al., 2015b). Fourth, the quantitative nature of the luciferase detector used in incPRINT offers the possibility of structure/function analyses of mutated or disordered RNA-protein interactions. Finally, incPRINT is flexible in its throughput: it was employed here a customized library of 3000 human test proteins representing the majority of all known RBPs, transcription factors and chromatin modifiers, however, the protein library can be conveniently expanded to include a range of additional proteins or reduced and customized to fit the experimental design and need.

Applying the incPRINT method to other noncoding or coding RNAs of various structural complexities will be an invaluable tool to dissect the precise mechanisms of RNA cellular functions through systematic identification of their binding proteins.

### REFERENCES

Almeida, M., Pintacuda, G., Masui, O., Koseki, Y., Gdula, M., Cerase, A., Brown, D., Mould, A., Innocent, C., Nakayama, M., et al. (2017). PCGF3/5-PRC1 initiates Polycomb recruitment in X chromosome inactivation. Science 356, 1081-1084.

Baltz, A. G., Munschauer, M., Schwanhäusser, B., Vasile, A., Murakawa, Y., Schueler, M., Youngs, N., Penfold-Brown, D., Drew, K., Milek, M., et al. (2012). The mRNA-bound proteome and its global occupancy profile on protein-coding transcripts. Molecular Cell 46, 674-690.

Bechara, E. G., Sebestyen, E., Bernardis, I., Eyras, E., and Valcárcel, J. (2013). RBM5, 6, and 10 Differentially Regulate NUMB Alternative Splicing to Control Cancer Cell Proliferation. Molecular Cell 52, 720-733.

Beletskii, A., Hong, Y. K., Pehrson, J., Egholm, M., and Strauss, W. M. (2001). PNA interference mapping demonstrates functional domains in the noncoding RNA Xist. Proceedings of the National Academy of Sciences 98, 9215-9220.

Borensztein, M., Syx, L., Ancelin, K., Diabangouaya, P., Picard, C., Liu, T., Liang, J.-B., Vassilev, I., Galupa, R., Servant, N., et al. (2017). Xist-dependent imprinted X inactivation and the early developmental consequences of its failure. Nature Structural & Molecular Biology 24, 226-233.

Borsani, G., Tonlorenzi, R., Simmler, M. C., Dandolo, L., Arnaud, D., Capra, V., Grompe, M., Pizzuti, A., Muzny, D., Lawrence, C., et al. (1991). Characterization of a murine gene expressed from the inactive X chromosome. Nature 351, 325-329.

Brown, C. J., Ballabio, A., Rupert, J. L., Lafrenière, R. G., Grompe, M., Tonlorenzi, R., and Willard, H. F. (1991). A gene from the region of the human X inactivation centre is expressed exclusively from the inactive X chromosome. Nature 349, 38-44.

Buzin, C. H., Mann, J. R., and Singer-Sam, J. (1994). Quantitative RT-PCR assays show Xist RNA levels are low in mouse female adult tissue, embryos and embryoid bodies. Development 120, 3529-3536.

Castello, A., Fischer, B., Eichelbaum, K., Horos, R., Beckmann, B. M., Strein, C., Davey, N. E., Humphreys, D. T., Preiss, T., Steinmetz, L. M., et al. (2012). Insights into RNA Biology from an Atlas of Mammalian mRNA-Binding Proteins. Cell 149, 1393-1406.

Chao, J. A., Patskovsky, Y., Almo, S. C., and Singer, R. H. (2007). Structural basis for the coevolution of a viral RNA-protein complex. Nature Structural & Molecular Biology 15, 103-105.

Chaumeil, J., Augui, S., Chow, J. C., and Heard, E. (2008). Combined immunofluorescence, RNA fluorescent in situ hybridization, and DNA fluorescent in situ hybridization to study chromatin changes, transcriptional activity, nuclear organization, and X-chromosome inactivation. Methods Mol. Biol. 463, 297-308.

Chu, C., Spitale, R. C., and Chang, H. Y. (2015a). Technologies to probe functions and mechanisms of long noncoding RNAs. Nature Structural & Molecular Biology 22, 29-35.

Chu, C., Zhang, Q. C., da Rocha, S. T., Flynn, R. A., Bharadwaj, M., Calabrese, J. M., Magnuson, T., Heard, E., and Chang, H. Y. (2015b). Systematic Discovery of Xist RNA Binding Proteins. Cell 161, 404-416.

da Rocha, S. T., and Heard, E. (2017). Novel players in X inactivation: insights into Xist-mediated gene silencing and chromosome conformation. Nature Publishing Group 24, 197-204.

da Rocha, S. T., Boeva, V., Escamilla-Del-Arenal, M., Ancelin, K., Granier, C., Matias, N. R., Sanulli, S., Chow, J., Schulz, E., Picard, C., et al. (2014). Jarid2 Is Implicated in the Initial Xist-Induced Targeting of PRC2 to the Inactive X Chromosome. Molecular Cell 53, 301-316.

Derrien, T., Johnson, R., Bussotti, G., Tanzer, A., Djebali, S., Tilgner, H., Guernec, G., Martin, D., Merkel, A., Knowles, D. G., et al. (2012). The GENCODE v7 catalog of human long noncoding RNAs: Analysis of their gene structure, evolution, and expression. Genome Research 22, 1775-1789.

Engreitz, J. M., Ollikainen, N., and Guttman, M. (2016). Long non-coding RNAs: spatial amplifiers that control nuclear structure and gene expression. Nature Publishing Group 17,756-770.

Guelman, S., Kozuka, K., Mao, Y., Pham, V., Solloway, M. J., Wang, J., Wu, J., Lill, J. R., and Zha, J. (2009). The Double-Histone-Acetyltransferase Complex ATAC Is Essential for Mammalian Development. Molecular and Cellular Biology 29, 1176-1188.

Guttman, M., and Rinn, J. L. (2012). Modular regulatory principles of large non-coding RNAs. Nature 482, 339-346.

Hasegawa, Y., Brockdorff, N., Kawano, S., Tsutui, K., Tsutui, K., and Nakagawa, S. (2010). The Matrix Protein hnRNP U Is Required for Chromosomal Localization of Xist RNA. Devcel 19, 469-476.

Hiriart, E., Gruffat, H., Buisson, M., Mikaelian, I., Keppler, S., Meresse, P., Mercher, T., Bernard, O. A., Sergeant, A., and Manet, E. (2005). Interaction of the Epstein-Barr virus mRNA export factor EB2 with human Spen proteins SHARP, OTT1, and a novel member of the family, OTT3, links Spen proteins with splicing regulation and mRNA export. Journal of Biological Chemistry 280, 36935-36945.

Jeon, Y., and Lee, J. T. (2011). YY1 Tethers Xist RNA to the Inactive X Nucleation Center. Cell 146, 119-133.

Kim, J. H., Lee, S.-R., Li, L.-H., Park, H.-J., Park, J.-H., Lee, K. Y., Kim, M.-K., Shin, B. A., and Choi, S.-Y. (2011). High Cleavage Efficiency of a 2A Peptide Derived from Porcine Teschovirus-1 in Human Cell Lines, Zebrafish and Mice. PLoS ONE 6, e18556-e18558.

Kittler, R., Surendranath, V., Heninger, A.-K., Slabicki, M., Theis, M., Putz, G., Franke, K., Caldarelli, A., Grabner, H., Kozak, K., et al. (2007). Genome-wide resources of endoribonuclease-prepared short interfering RNAs for specific loss-of-function studies. Nat Meth 2, 181-188.

Konig, J., Zarnack, K., Luscombe, N. M., and Ule, J. (2012). Protein-RNA interactions: new genomic technologies and perspectives. Nat. Rev. Genet. 13, 77-83.

Lu, Z., Zhang, Q. C., Lee, B., Flynn, R. A., Smith, M. A., Robinson, J. T., Davidovich, C., Gooding, A. R., Goodrich, K. J., Mattick, J. S., et al. (2016). RNA Duplex Map in Living Cells Reveals Higher-Order Transcriptome Structure. Cell 1-14.

McHugh, C. A., Chen, C.-K., Chow, A., Surka, C. F., Tran, C., McDonel, P., Pandya-Jones, A., Blanco, M., Burghard, C., Moradian, A., et al. (2015). The Xist lncRNA interacts directly with SHARP to silence transcription through HDAC3. Nature 521, 232-236.

Mili, S., and Steitz, J. A. (2004). Evidence for reassociation of RNA-binding proteins after cell lysis: implications for the interpretation of immunoprecipitation analyses. Rna 10,1692-1694.

Minajigi, A., Froberg, J. E., Wei, C., Sunwoo, H., Kesner, B., Colognori, D., Lessing, D., Payer, B., Boukhali, M., Haas, W., et al. (2015a). A comprehensive Xist interactome reveals cohesin repulsion and an RNA-directed chromosome conformation. Science 1-19.

Minajigi, A., Froberg, J. E., Wei, C., Sunwoo, H., Kesner, B., Colognori, D., Lessing, D., Payer, B., Boukhali, M., Haas, W., et al. (2015b). A comprehensive Xist interactome reveals cohesin repulsion and an RNA-directed chromosome conformation. Science 1-19.

Moindrot, B., and Brockdorff, N. (2016). RNA binding proteins implicated in Xist-mediated chromosome silencing. Semin. Cell Dev. Biol. 1-13.

Moindrot, B., Cerase, A., Coker, H., Masui, O., Grijzenhout, A., Pintacuda, G., Schermelleh, L., Nesterova, T. B., and Brockdorff, N. (2015). A Pooled shRNA Screen Identifies Rbm15, Spen, and Wtap as Factors Required for Xist RNA-Mediated Silencing. CellReports 12, 562-572.

Monfort, A., Di Minin, G., Postlmayr, A., Freimann, R., Arieti, F., Thore, S., and Wutz, A. (2015). Identification of Spen as a Crucial Factor for Xist Function through Forward Genetic Screening in Haploid Embryonic Stem Cells. CellReports 12, 554-561.

Nesterova, T. B., Slobodyanyuk, S. Y., Elisaphenko, E. A., Shevchenko, A. I., Johnston, C., Pavlova, M. E., Rogozin, I. B., Kolesnikov, N. N., Brockdorff, N., and Zakian, S. M. (2001). Characterization of the genomic Xist locus in rodents reveals conservation of overall gene structure and tandem repeats but rapid evolution of unique sequence. Genome Research 11, 833-849.

Patil, D. P., Chen, C.-K., Pickering, B. F., Chow, A., Jackson, C., Guttman, M., and Jaffrey, S. R. (2016). m6A RNA methylation promotes XIST-mediated transcriptional repression. Nature 537, 369-373.

Patrat, C., Okamoto, I., Diabangouaya, P., Vialon, V., Le Baccon, P., Chow, J., and Heard, E. (2009). Dynamic changes in paternal X-chromosome activity during imprinted X-chromosome inactivation in mice. Proc. Natl. Acad. Sci. U.S.a. 106, 5198-5203.

Pinheiro, I., and Heard, E. (2017). X chromosome inactivation: new players in the initiation of gene silencing. F1000Res 6, 344-10.

Ran, F. A., Hsu, P. D., Wright, J., Agarwala, V., Scott, D. A., and Zhang, F. (2013). Genome engineering using the CRISPR-Cas9 system. Nat Protoc 8,2281-2308.

Ridings-Figueroa, R., Stewart, E. R., Nesterova, T. B., Coker, H., Pintacuda, G., Godwin, J., Wilson, R., Haslam, A., Lilley, F., Ruigrok, R., et al. (2017). The nuclear matrix protein CIZ1 facilitates localization of Xist RNA to the inactive X-chromosome territory. Genes & Development 31, 876-888.

Riley, K. J., Yario, T. A., and Steitz, J. A. (2012). Association of Argonaute proteins and microRNAs can occur after cell lysis. Rna 18, 1581-1585.

Sarma, K., Cifuentes-Rojas, C., Ergun, A., del Rosario, A., Jeon, Y., White, F., Sadreyev, R., and Lee, J. T. (2014). ATRX Directs Binding of PRC2 to Xist RNA and Polycomb Targets. Cell 159, 869-883.

Sarma, K., Levasseur, P., Aristarkhov, A., and Lee, J. T. (2010). Locked nucleic acids (LNAs) reveal sequence requirements and kinetics of Xist RNA localization to the X chromosome. Proc. Natl. Acad. Sci. U.S.a. 107, 22196-22201.

Savarese, F., Flahndorfer, K., Jaenisch, R., Busslinger, M., and Wutz, A. (2006). Hematopoietic Precursor Cells Transiently Reestablish Permissiveness for X Inactivation. Molecular and Cellular Biology 26, 7167-7177.

Schulz, E. G., Meisig, J., Nakamura, T., Okamoto, I., Sieber, A., Picard, C., Borensztein, M., Saitou, M., Blüthgen, N., and Heard, E. (2014). The Two Active X Chromosomes in Female ESCs Block Exit from the Pluripotent State by Modulating the ESC Signaling Network. Stem Cell 14, 203-216.

Senner, C. E., Nesterova, T. B., Norton, S., Dewchand, H., Godwin, J., Mak, W., and Brockdorff, N. (2011). Disruption of a conserved region of Xist exon 1 impairs Xist RNA localisation and X-linked gene silencing during random and imprinted X chromosome inactivation. Development 138, 1541-1550.

Sunwoo, H., Colognori, D., Froberg, J. E., Jeon, Y., and Lee, J. T. (2017). Repeat E anchors Xist RNA to the inactive X chromosomal compartment through CDKN1A-interacting protein (CIZ1). Proc. Natl. Acad. Sci. U.S.a. 114, 10654-10659.

Taipale, M., Krykbaeva, I., Koeva, M., Kayatekin, C., Westover, K. D., Karras, G. I., and Lindquist, S. (2012). Quantitative analysis of HSP90-client interactions reveals principles of substrate recognition. Cell 150, 987-1001.

Van Nostrand, E. L., Pratt, G. A., Shishkin, A. A., Gelboin-Burkhart, C., Fang, M. Y., Sundararaman, B., Blue, S. M., Nguyen, T. B., Surka, C., Elkins, K., et al. (2016). Robust transcriptome-wide discovery of RNA-binding protein binding sites with enhanced CLIP (eCLIP). Nat Meth 13, 508-514.

Wutz, A., Rasmussen, T. P., and Jaenisch, R. (2002). Chromosomal silencing and localization are mediated by different domains of Xist RNA. Nat. Genet. 30, 167-174.

Zhao, J., Sun, B. K., Erwin, J. A., Song, J.-J., and Lee, J. T. (2008a). Polycomb proteins targeted by a short repeat RNA to the mouse X chromosome. Science 322, 750-756.

Zhao, J., Sun, B. K., Erwin, J. A., Song, J.-J., and Lee, J. T. (2008b). Polycomb proteins targeted by a short repeat RNA to the mouse X chromosome. Science 322, 750-756.

1. A method for detecting the interaction in a cell between an RNA and a protein, wherein the cell expresses :
the RNA fused to a hairpin, called hairpin-tagged RNA,
the protein fused to a tag, called tagged-protein,
a reporter protein fused to a protein which binds to said hairpin,
wherein said reporter protein is a luminescent reporter protein, said method comprises the lysis of the cell and the detection of the complex tagged protein/hairpin-tagged RNA/hairpin binding protein—reporter protein.

2. The method according to claim 1, wherein the detection of the complex tagged protein/hairpin-tagged RNA/hairpin binding protein—reporter protein comprises the detection of the tag of the tagged protein and the detection of the reporter protein.

3. The method according to claim 1, wherein the detection of both the tag and the reporter protein indicates the interaction between the RNA and the protein.

4. The method according to claim 1, wherein the reporter protein is an enzyme which catalyzed a bioluminescence reaction, or a fluorescent protein.

5. The method according to claim 1, wherein the reporter protein is a luciferase.

6. The method according to claim 1, wherein the hairpin used to tag the RNA is selected from the group consisting in: MS2, PP7, AN, TAR, iron responsive elements (IREs) and U1A hpII.

7. The method according to claim 1, wherein the RNA is tagged with between 2 to 24 consecutive identical hairpins.

8. The method according to claim 1, wherein the hairpin binding protein is selected from the group consisting in: MS2CP (MS2 coat protein), PP7CP (PP7 coat protein), Qβ, GA, BoxB, TAT, IRP and U1A.

9. The method according to claim 1, wherein the hairpin used to tag the RNA and the binding protein of said hairpin are selected from the couples hairpin/binding protein consisting in: MS2/MS2CP, PP7/PP7CP, MS2/Q13, MS2/GA, AN/BoxB, TAR/TAT, iron responsive elements (IREs)/IRP, and U1A hpII/U1A.

10. The method according to claim 1, wherein the tag for the tagged-protein is selected from the group consisting in: FLAG, HIS, CBP, HA, Myc, poly His, V5.

11. The method according to claim 1, wherein the protein is fused to a plurality of identical consecutive tags.

12. A method for detecting the effect of a compound on the interaction in a cell between an RNA and a protein, said method being carried out according to claim 1, separately into a cell cultivated in the presence of a compound to be tested and into a cell cultivated in the absence of said compound in the culture medium.

13. A method for determining the effect of the RNA methylation on the interaction between an RNA and a protein, wherein said method being carried out according to claim 1, separately into a cell having a normal profile for the RNA methylation and into a cell comprising the inactivation of one or more genes involved in the RNA methylation.

14. A method for detecting the interaction in a cell between two RNAs and a protein, said method comprises two hairpin-tagged RNAs, each of the RNAs being fused to a different hairpin, and wherein said cell expresses two different reporter proteins respectively fused to two different proteins which respectively bind to said hairpins, said method being carried out according to claim 1.

15. The method according to claim 15, wherein the two different RNA are two versions of the same transcript, one is the RNA normally expressed by cells, and the other a mutated version of said RNA.
